SEQUENCE LISTING (21 sequences)

SEQ ID NO: 1 - DNA, 27 bp, Artificial Sequence
  Description of Artificial Sequence Synthetic primer
ttttttggatcccagttgcataaagat

SEQ ID NO: 2 - DNA, 27 bp, Artificial Sequence
  Description of Artificial Sequence Synthetic primer
ttttttgtcgactatccgattagggtt

SEQ ID NO: 3 - DNA, 19 bp, Artificial Sequence
  Description of Artificial Sequence Synthetic primer
gggtacctcatgtgacaag

SEQ ID NO: 4 - DNA, 20 bp, Artificial Sequence
  Description of Artificial Sequence Synthetic primer
tcacttcttcactggtcatg

SEQ ID NO: 5 - DNA, 23 bp, Artificial Sequence
  Description of Artificial Sequence Synthetic primer
atgaatcctacactcatccttgc

SEQ ID NO: 6 - DNA, 26 bp, Artificial Sequence
  Description of Artificial Sequence Synthetic primer
tcacacagtggggtagctggctgctg

SEQ ID NO: 7 - DNA, 22 bp, Artificial Sequence
  Description of Artificial Sequence Synthetic primer
atgtgggggctcaaggttctgc

SEQ ID NO: 8 - DNA, 22 bp, Artificial Sequence
  Description of Artificial Sequence Synthetic primer
tcacatcttggggaagctggcc

SEQ ID NO: 9 - DNA, 23 bp, Artificial Sequence
  Description of Artificial Sequence Synthetic primer
atgaatctttcgctcgtcctggc

SEQ ID NO: 10 - DNA, 21 bp, Artificial Sequence
  Description of Artificial Sequence Synthetic primer
tcacacattggggtagctggc

SEQ ID NO: 11 - DNA, 32 bp, Artificial Sequence
  Description of Artificial Sequence Synthetic primer
atctggcaccacaccttctacaatgagctgcg

SEQ ID NO: 12 - DNA, 32 bp, Artificial Sequence
  Description of Artificial Sequence Synthetic primer
cgtcatactcctgcttgctgatccacatctgc

SEQ ID NO: 13 - protein, 118 aa, Homo sapiens
MRGSHHHHHHGSQLHKDPTLDHHWHLWKKTYGKQYKEKNEEAVRRLIWEKNLKFVMLHNLEHSMGMHSYDLGMNHLGDMTSEEVMSLTSSLRVPSQWQRNITYKSNPNRVDLQPSLIS

SEQ ID NO: 14 - DNA, 357 bp, Homo sapiens
  CDS (1)..(354)
atgagaggatcgcatcaccatcaccatcacggatcccagttgcataaagatcctaccctggatcaccactggcatctctggaagaaaacctatggcaaacaatacaaggaaaagaatgaagaagcagtacgacgtctcatctgggaaaagaatctaaagtttgtgatgcttcacaacctggagcattcaatgggaatgcactcatacgatctgggcatgaaccacctgggagacatgaccagtgaagaagtgatgtctttgacgagttccctgagagttcccagccagtggcagagaaatatcacatataagtcaaaccctaatcgggtcgacctgcagccaagcttaattagctga

SEQ ID NO: 15 - DNA, 357 bp, Homo sapiens
tcagctaattaagcttggctgcaggtcgacccgattagggtttgacttatatgtgatatttctctgccactggctgggaactctcagggaactcgtcaaagacatcacttcttcactggtcatgtctcccaggtggttcatgcccagatcgtatgagtgcattcccattgaatgctccaggttgtgaagcatcacaaactttagattcttttcccagatgagacgtcgtactgcttcttcattcttttccttgtattgtttgccataggttttcttccagagatgccagtggtgatccagggtaggatctttatgcaactgggatccgtgatggtgatggtgatgcgatcctctcat

SEQ ID NO: 16 - DNA, 32 bp, Artificial Sequence
  Description of Artificial Sequence Synthetic primer
ttttttggatcccagttgcataaagatcctac

SEQ ID NO: 17 - DNA, 27 bp, Artificial Sequence
  Description of Artificial Sequence Synthetic primer
ttttttgtcgacccgattagggtttga

SEQ ID NO: 18 - protein, 6 aa, Artificial Sequence
  Description of Artificial Sequence Synthetic 6xHis tag
HHHHHH

SEQ ID NO: 19 - DNA, 1763 bp, Homo sapiens
ggggtacctcatgtgacaagttccaatttcttttcaagtcaattgaactgaaatctccttgttgctttgaaatcttagaagagagcccactaattcaaggactcttactgtaggagcaactgctggttctatcacaatgaaacggctggtttgtgtgctcttggtgtgctcctctgcagtggcacagttgcataaagatcctaccctggatcaccactggcatctctggaagaaaacctatggcaaacaatacaaggaaaagaatgaagaagcagtacgacgtctcatctgggaaaagaatctaaagtttgtgatgcttcacaacctggagcattcaatgggaatgcactcatacgatctgggcatgaaccacctgggagacatgaccagtgaagaagtgatgtctttgatgagttccctgagagttcccagccagtggcagagaaatatcacatataagtcaaaccctaatcggatattgcctgattctgtggactggagagagaaagggtgtgttactgaagtgaaatatcaaggttcttgtggtgcttgctgggctttcagtgctgtgggggccctggaagcacagctgaagctgaaaacaggaaagctggtgtctctcagtgcccagaacctggtggattgctcaactgaaaaatatggaaacaaaggctgcaatggtggcttcatgacaacggctttccagtacatcattgataacaagggcatcgactcagacgcttcctatccctacaaagccatggatctgaaatgtcaatatgactcaaaatatcgtgctgccacatgttcaaagtacactgaacttccttatggcagagaagatgtcctgaaagaagctgtggccaataaaggcccagtgtctgttggtgtagatgcgcgtcatccttctttcttcctctacagaagtggtgtctactatgaaccatcctgtactcagaatgtgaatcatggtgtacttgtggttggctatggtgatcttaatgggaaagaatactggcttgtgaaaaacagctggggccacaactttggtgaagaaggatatattcggatggcaagaaataaaggaaatcattgtgggattgctagctttccctcttacccagaaatctagaggatctctcctttttataacaaatcaatgaaatatgaagcactttctcttaacttaatttttcctgctgtatccagaagaaataattgtgtcatgattaatgtgtatttactgtactaattagaaaatatagtttgaggccgggcacgtggctcacgcgtaatcccgttacttgggaggccaaggcaggcattatcaatcttgaggccaggagttaaagagcagcctggctaacatggtgaaaccccatctctactaaaaatacaaaaaattagccgagcacggtggtgcatgcctgtaatcccagctacttgggaggctgaggcacgagattccttgaacccaagaggttgaggctatgttgagctgagatcacaccactgtactccagcctggatgacagagtggagactctgtttcaaaaaaacagaaaagaaaatatagtttgattcttcatttttttaaatttgcaaatctcaggataaagtttgctaagtaaattagtaatgtactatagatataactgtacaaaaattgttcaacctaaaacaatctgtaattgcttattgttttattgtcccgaattcagttggtttaatatattgtcctctgtaatttcgatccttcttaa

SEQ ID NO: 20 - protein, 331 aa, Homo sapiens [sequence omitted]

SEQ ID NO: 21 - protein, 118 aa, Homo sapiens
MRGSHHHHHHGSQLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRVDLQPSLIS

(19) United States
(12) Patent Application Publication
(10) Pub. No.: US 2010/0104554 A1
(43) Pub. Date: Apr. 29, 2010

(54) PEPTIDE AND USES THEREOF

(75) Inventors: Christopher Scott, Belfast (GB); Roberta Burden, Belfast (GB); Jim Johnston, Belfast (GB); Mark McCurley, Belfast (GB); Philip Snoddy, Belfast (GB); Richard Buick, Belfast (GB)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: Fusion Antibodies Limited, a Corporation of Great Britian, Belfast (GB)

(21) Appl. No.: 12/281,356

(22) PCT Filed: Mar. 2, 2007
(86) PCT No.: PCT/GB07/00744

§ 371 Date: Jan. 16, 2009

(30) Foreign Application Priority Data
Mar. 2, 2006 (GB) GB0604187.5

(51) Int. Cl. A61K 39/395; A61K 38/48; C12P 21/06; C12N 9/64; A61K 38/00; A61K 31/7088

(52) U.S. Cl. 424/130.1; 424/94.63; 435/69.1; 435/226; 514/2; 514/44 R

(57) ABSTRACT

A method of inhibiting activity of a cathepsin L-like protease in cells or tissue and the use of the method in the treatment of disease such as cancer and inflammatory diseases is described. The method comprises administration of a cathepsin propeptide or a nucleic acid encoding a cathepsin propeptide. In particular embodiments, the propeptide is a Cathepsin S propeptide. Further, the use of propeptides having an Fc portion is described.

### FIELD OF THE INVENTION

This application relates to a peptide and its use in methods of treatment. In particular, it relates to a cathepsin propeptide, methods of its production and uses of the propeptide.

### BACKGROUND TO THE INVENTION

Proteases are a large group of proteins that comprise approximately 2% of all gene products (Rawlings and Barrett, 1999). Proteases catalyse the hydrolysis of peptide bonds and are vital for the proper functioning of all cells and organisms. Proteolytic processing events are important in a wide range of cellular processes including bone formation, wound healing, angiogenesis and apoptosis.

The lysosomal cysteine proteases were initially thought to be enzymes that were, responsible for non-selective degradation of proteins in the lysosomes. Normally associated with localisation in the lysosomes, these proteases were originally thought to be only involved in the non-selective degradation of proteins in endosomal compartments. However, they are now known to be accountable in a number of specific cellular processes, having roles in antigen presentation (Honey and Rudensky, 2003; Bryant & Ploegh, 2004) apoptosis (Zheng et al, 2005; Broker et al, 2005), pro-hormone processing (Hook et al, 2004) and extracellular matrix remodelling (Chapman et al, 1994; Chapman et al, 1997).

Cathepsins are proteolytic enzymes. To date, eleven human cathepsins have been identified, but the specific in vivo roles of each are still to be determined (Katunuma et al, 2003). Cathepsins B, L, H, F, O, X and C are expressed in most cells, suggesting a possible role in regulating protein turnover, whereas cathepsins S, K, W and V are restricted to particular cells and tissues, indicating that they may have more specific roles (Kos et al, 2001; Berdowska, 2004). Cathepsin L-like proteases (which include CatL, S and K) are proteolytic enzymes which belong to the CA clan of cysteine proteases. Each of these lysosomal proteases has been implicated in the progression of various tumours. It is thought that their abnormally high secretion from tumour cells leads to the degradation of the extracellular matrix (ECM). This aberrant breakdown of ECM components such as elastin and collagen accelerates the penetration and invasion of these abnormal cells to surrounding normal tissue.

Cathepsin L-like proteases are produced as inactive precursors; containing an N terminal propeptide domain. This propeptide has previously been shown to act as both as a chaperone for the folding of the nascent protease and inhibitor of the active species, binding to the active site of the protease in immature lysosomes. Inhibition studies have shown that the CatS propeptide (CatSPP) has a Kᵢ in the low nanomolar range towards activated CatS and perhaps surprisingly, also has similar properties against both CatK and CatL, although it has also been shown to have no effect on the less homologous CatB, CatH or papain. Moreover, this property of the CatSPP is unique in that the propeptides of K and L dp not have the same uniform inhibition profile to each of its cognate family members.

Cat S (Cathepsin S) was originally identified from bovine lymph nodes and spleen and the human forth cloned from a human macrophage cDNA library (Shi et al, 1992). The gene encoding Cat S is located on human chromosome 1q21. The 996 base pair transcript encoded by the Cat S gene is initially translated into an unprocessed precursor protein with a molecular weight of 37.5 kDa. The unprocessed protein is composed of 331 amino acids; a 15 amino acid signal peptide, a 99 amino acid pro-peptide sequence and a 217 amino acid peptide. Cat S is initially expressed with a signal peptide that is removed after it enters the lumen of the endoplasmic reticulum. The propeptide sequence binds to the active site of the protease, rendering it inactive until it has been transported to the acidic endosomal compartments, after which the propeptide sequence is removed and the protease is activated (Baker et al, 2003).

CatS has been identified as a key enzyme in major histocompatibility complex class II (MHC-II) mediated antigen presentation, by cleavage of the invariant chain, prior to antigen loading. Studies have shown that mice deficient in Cat S have an impaired ability to present exogenous proteins by APC's (Nakagawa et al, 1999). The specificity of Cat S in the processing of the invariant chain Ii, allows for Cat S specific therapeutic targets in the treatment of conditions such as asthma and autoimmune disorders (Chapman et al, 1997).

Cathepsin L was originally isolated from the lysosomes of rat liver before the human form was identified in 1988 (Gal and Gottesman, 1988; Joseph et al, 1988). The gene encoding CatL was mapped to human chromosome 9q21-22 (Fan et al., 1989; Chauhan et al., 1993) and is composed of eight exons and seven introns. The gene product is translated into a preproprotein with a molecular weight of 39 kDa and is processed into two enzymatically active isoforms; a single chain form of 31 kDa and a two-chain form comprised of a 24 kDa heavy chain and a 5 kDa light chain (Mason et al 1989). The processing of pro-CatL to the mature active enzyme can occur via various mechanisms including autocatalytic activation (Salminen & Gottesman, 1990) and by the action of CatD (Nishimura et al., 1989; Wiederanders & Kirschke, 1989) or metallo-endopeptidases (Hara et al., 1988).

CatL has endopeptidase activity, and preferentially cleaves peptide bonds with hydrophobic amino acid residues in the P2 and P3 positions (Kärgel et al., 1980, 1981). It has been shown to hydrolyze several proteins with the same specific activity as cathepsin S (Kirschke et al., 1989). However, it favours aromatic residues in the P2 position, distinguishing itself from the closely related cathepsins S and K (McGrath, 1999).

CatL has been proposed to have a major role in many biological processes including lysosomal proteolysis and bone resorption, as well as in several diseases such as arthritis and malignancy (Rukamp and Powers, 2002). The role of lysosomal cysteine proteases in antigen presentation has been extensively researched within the past few years. CatL has been implicated in this process through its ability to perform the final step of Ii proteolysis in cortical thymic epithelial cells. Further evidence has shown that the p41 isoform of the Ii chain has the ability to interact with the mature CatL protein, inhibiting its activity and stabilising it in neutral pH environments (Ogrinc et al, 1993; Bevec et al, 1996). Studies on CatL-deficient mice were observed to be incapable of the degradation of the invariant chain in cortical epithelial cells of the thymus (Nakagawa et al, 1998) and exhibited a distinct defect in CD4+ T cell selection (Roth et al, 2000). Mice lacking cathepsin L also developed periodic hair loss and epidermal hyperplasia due to alterations in hair follicle morphogenesis.

The role of CatL in tumour invasion and metastasis has also been studied in great detail due to its ubiquitous expression and its ability to degrade components of the extracellular matrix and basement membrane. Elevated expression levels of CatL have been associated with a wide range of malignancies including breast, colon, prostate, kidney carcinomas and astrocytomas.

Recent evidence has also suggested that CatL may function as a transcriptional activator. Alternative isoforms of CatL have previously been reported (Rescheleit et al, 1996; Seth et al, 2003), however an isoform lacking the N-terminal signal peptide has been shown to localise to the nucleus, suggesting a role for CatL in the processing of the CDP/Cux transcription factor. This theory was reinforced by studies on CatL-deficient fibroblasts, which appeared to have a marked reduction in CDP/Cux processing (Goulet et al, 2004).

Cathepsin K was first cloned from cDNA rabbit in 1994 (Tezuka et al, 1994), prior to the description of the human ortholog the following year by several independent groups (Bromine et al, 1995; Shi et al, 1995; Inaoka et al, 1995). The gene encoding CatK is situated on human chromosome 1q21, the same locus as CatS, suggesting that these two proteases may have a common origin. The promoter structure of CatK is similar to that of CatS with the absence of a TATA box but with the presence of two AP-1 sites; both common features of genes which show restricted expression patterns. Human CatK expression has been shown to be restricted and is found predominantly in osteoclasts and in the ovary (Bromine et al, 1995; Drake et al, 1996).

The amino acid sequence of CatK shows high sequence similarity with cathepsins S and L (52% and 46% respectively) and together these three genes form a small subfamily within the mammalian lysosomal cysteine proteases. CatK has been characterised as one of the most potent elastinolytic enzymes, with greater activity that pancreatic elastase at pH5.5 (Bromme et al, 1996; Chapman et al, 1997). It also has the ability to catalyse the hydrolysis of collagen type I, II and IV (Kafienah et al, 0.1998).

The physiological relevance of the collagenolytic activity of CatK is illustrated through its association with the bone disorder, pycnodysostosis (Gelb et al, 1996). Pycnodysostosis is ah autosomal recessive condition characterised by osteosclerosis and severe skeletal dysplasia. Osteoporosis occurs when the balance between bone resorption and formation has been disrupted, favouring resorption. Resorption is mediated by osteoclasts which generate an acidic environment at their site of attachment where the proteolytic degradation of the matrix occurs. CatK has been implicated in this process due to the identification of nonsense, missense and stop codon mutations in pycnodysostosis patients (Gelb et al, 1996). CatK knockout mice also exhibit a decreased matrix degrading activity in their osteoclasts, however the murine phenotype is less severe than in the human condition (Saftig et al, 1998).

CatK expression appears to be upregulated at sites of inflammation and by retinoic acid in osteoclastic cells lines (Saneshige et al, 1995). Its expression has been detected in giant cell tumours of the bone, prostate and breast carcinomas (Brubaker et al, 2003; Littlewood-Evans et al, 1997) as well as in the synovial fibroblasts of patients with rheumatoid arthritis (Hummel et al, 1998).

Cathepsin V was first identified from a human brain cDNA library as a cysteine protease with exceptionally high homology to CatL (78%) (Santamaria et al., 1998). Moreover, the gene encoding CatV has been mapped to human chromosome 9q21-22, adjacent to CatL. The high homology and close proximity between the CatL and V genes suggests that the two proteases may have evolved from a common ancestral precursor (Itoh et al., 1999; Bromine et al., 1999). However, the widespread expression pattern observed with CatL has not been mimicked by CatV, with expression restricted to the thymus, testis and corneal epithelium (Adachi et al, 1998, Bromme et al, 1999, Tolosa et al, 2003). The restricted tissue expression of this protease is indicative of specialised function and it is thought that CatV is essential in MHC class II antigen presentation in specific cell types (Shi et al., 1999; Tolosa et al., 2003). Sequence alignment with other human cathepsins has placed CatV in the same phylogenetic branch of human C1 peptidases as CatL, S and K (Buhling et al., 2000).

### Pathological Association of Cathepsins

The alterations in protease expression patterns underlie many human pathological processes. The deregulated expression and activity of cathepsins, has been linked to a range of conditions including neurodegenerative disorders, autoimmune diseases and tumourigenesis.

Cat S upregulation has been linked to several neurodegenerative disorders. It is believed to have a role in the production of the β peptide (Aβ) from the amyloid precursor protein (APP) (Munger et al, 1995) and its expression has been shown to be upregulated in both Alzheimer's Disease and Down's Syndrome (Lemere et al, 1995). Cat S may also have a role in Multiple Sclerosis and Creutzfeldt-Jakob disease through the ability of Cat S to degrade myelin basic protein, a potential autoantigen implicated in the pathogenesis of MS (Beck et al, 2001) and in CJD patients, Cat S expression has been shown to increase more than four fold (Baker et al, 2002).

Aberrant Cat S expression has also been associated with atherosclerosis. Cat S expression is negligible in normal arteries, yet human atheroma display strong immunoreactivity (Sukhova et al, 1998). Further studies using knockout mice, deficient in both Cat S and the LDL-receptor, were shown to develop significantly less atherosclerosis (Sukhova et al, 2003). Further research has linked Cat S expression with inflammatory muscle disease and rheumatoid arthritis. Muscle biopsy specimens from patients with inflammatory myopathy had a 10 fold increase in Cat S expression compared to control muscle sections (Wiendl et al, 2003), and levels of Cat S expression were significantly higher in synovial fluid from patients with rheumatoid arthritis compared to those with osteoarthritis (Hashimoto et al, 2001).

The role of Cat S has also been investigated in specific malignancies. The expression of Cat S was shown to be significantly greater in lung tumour and prostate carcinomas sections in comparison to normal tissue (Kos et al, 2001, Fernandez et al, 2001) and suggested that Cat S may have a role in tumour invasion and disease progression.

Recent work in this laboratory on Cat S demonstrated the significance of its expression in human astrocytomas (Flannery et al, 2003; Flannery et al, 2006). Immunohistochemical analysis showed the expression of Cat S in a panel of astrocytoma biopsy specimens from WHO grades I to IV, but appeared absent from normal astrocytes, neurones, oligodendrocytes and endothelial cells. Cat S expression appeared highest in grade IV tumours and levels of extracellular activity were greatest in cultures derived from grade TV tumours.

Cat S has been shown to be active in the degradation of ECM macromolecules such as laminin, collagens, elastin and chondroitin sulphate proteoglycans (Liuzzo et al, 1999) and invasion assays using the U251MG grade IV glioblastoma cell line showed up to 61% reduction in invasion in the presence of a Cat S inhibitor LHVS29 (Flannery et al, 2003). This would suggest that Cat S may have an important role in the process of tumour invasion in astrocytomas and therefore may be a target for anti-invasive therapy.

CatL has also been found to have important roles in a range of different pathological conditions including tumourigenesis. The generation of CatL knockout mice revealed a critical role in epidermal homeostasis, regulation of the hair cycle, and MHC class II-mediated antigen presentation in cortical epithelial cells of the thymus (Reinheckel et al, 2001).

Cat K expression has previously been correlated with a range of different pathologies including osteoporosis and specific malignancies. The rare skeletal condition, pycnodysostosis is caused by a deficiency in CatK. CatK normally functions to degrade type-1 collagen and other bone proteins (Motyckova and Fisher, 2002) The osteoclasts from patients with Pycnodysostosis are dysfunctional due to mutations within the cathepsin K gene (Gelb et al, 1996).

CatK expression is associated with lung adenocarcinomas yet absent from the non-invasive bronchioalveolar carcinomas, acting as a potential marker of the invasive growth of lung carcinomas (Rapa et al, 2006). In addition, CatK has also been identified as the principal protease in giant cell tumour of the bone (Lindeman et al, 2004) and an association with breast carcinomas (Littlewood-Evans et al, 1997) has been shown. Therefore, the development of CatK inhibitors has great potential, particularly in pathological conditions where excess osteoclast activation and bone resorption occurs such as osteoporosis, bone metastasis and multiple myeloma.

Cat V was originally identified in colorectal and breast carcinomas, as well as certain ovarian and renal cell carcinomas as a cysteine protease with exceptionally high homology to CatL (78%) (Santamaria et al., 1998). Moreover, the gene for CatV has been mapped to human chromosome 9q21-22, adjacent to CatL. The high homology and close proximity of their encoding genes suggests that the two proteases may have evolved from a common ancestral precursor (Itoh et al., 1999; Bromme et al., 1999). However, although CatL has widespread tissue expression, CatV is normally restricted to the thymus, testis and corneal epithelium (Adachi et al. 1998; Bromme et al. 1999). The restricted tissue expression of this protease is indicative of specialised function and it is thought that CatV is essential in MHC class II antigen presentation in specific cell types (Shi et al. 1999; Tolosa et al., 2003).

The increase in expression and activity of the cathepsin L-like proteases has been observed in a range of diseases and implicated in their pathogenesis. Therefore, the generation of inhibitors specifically targeting these proteases have the potential as therapeutic agents.

### Inhibition of Cathepsin L-Like Proteases

When proteases are over-expressed, therapeutic strategies have focused on the development of inhibitors to block the activity of these enzymes. The generation of specific small molecule inhibitors to the cathepsins have proved difficult in the past, due to problems with selectivity and specificity. The dipeptide α-keto-β-aldehydes developed as potent reversible inhibitors to Cat S by Walker et al, had the ability to inhibit Cat B and L, albeit with less efficiency (Walker et al, 2000) and the Cat S inhibitor 4-Morpholineurea-Leu-HomoPhe-vinylsulphone (LHVS) has also been shown to inhibit other cathepsins when used at higher concentrations (Palmer et al, 1995).

The development of small molecule inhibitors for the CatL-like proteases, both reversible and irreversible, is well documented. The clinical application of such compounds is questionable due to poor specificity, inhibition of the proteases in normal tissues, and possible reactivity to bystander proteins (Turk et al, 2004). Therefore alternative strategies that could target only secreted proteolytic activities are attractive. Furthermore, inhibitors that have high selectivity for this sub-family of proteases, yet broad specificity within this group may prove more useful, due to the overlap in function that has been shown from gene knockout studies (Saftig et al, 1998; Nakagawa et al, 1998; Nakagawa et al, 1999).

Of all the characterised propeptides, CatSPP has the most interesting inhibitory kinetic profile, as it is an equally effective inhibitor of both CatL and CatK in addition to CatS. Maubach and co-workers showed in competitive enzyme binding assays that CatSPP is an equipotent inhibitor of CatS (Kᵢ of 0.27 nM) and CatL (Kᵢ of 0.36 nM) (Maubach et al., 1997), whereas more recent work suggests that CatSPP is actually a more potent inhibitor of CatL (Kᵢ of 0.46 nM) than it is of CatS (Kᵢ of 7.6 nM) and has almost identical efficacy against CatK (Kᵢ of 7.0 nM) (Guay et al, 2000).

As described above, on normal, activation of cathepsin, the natural propeptide undergoes a conformational change and is released. After release, the propeptide is presumed to be redundant.

### SUMMARY OF THE INVENTION

The present inventors have surprisingly shown that the exogenously applied cathepsin S propeptide (CatSPP) has a potent specific inhibitory action on the activity of cathepsin L-like proteases in invasive cancer models. This result was particularly unexpected given that it is thought that, once the cysteine cathepsin protease is activated in vivo, the remnant propeptide fragment is redundant and can no longer have, any effect on the protease. It was assumed that, under the same conditions in vivo, exogenously added propeptide would similarly have no effect. Moreover, given that the propeptide is basic in nature, trypsin-like activities present in and on the cell would be expected to break down any exogenous propeptides.

These, results indicate that, contrary to expectations, cathepsin propeptides may be used to attenuate the progression of invasive or metastatic cancer cells and thus may be used in a therapeutic context.

Accordingly, in a first aspect of the present invention, there is provided a method of inhibiting activity of a cathepsin L-like protease in cells or tissue, said method comprising administration of a cathepsin propeptide or a nucleic acid encodings cathepsin propeptide to said cells or tissue.

In one embodiment, the method is in vitro. In another the method is in vivo.

Activity may be inhibited completely or partially. Thus the method may be used to reduce aberrant activity to normal activity.

In a second aspect of the present invention, there is provided a method of inhibiting overexpression of a cathepsin L-like protease; in cells or tissue, said method comprising administration of a cathepsin propeptide or a nucleic acid encoding a cathepsin propeptide to said cells or tissue.

In a further aspect, there is provided a method of treating a condition, associated with overexpression and/or aberrant activity of a cathepsin L-like protease in a patient in need of treatment, thereof, said method comprising administration of a cathepsin propeptide or a nucleic acid encoding a cathepsin propeptide.

Further provided is a cathepsin propeptide or a nucleic acid encoding a cathepsin propeptide for use in medicine.

The invention further provides a cathepsin propeptide or a nucleic acid encoding a cathepsin propeptide for use in treatment of a condition associated with overexpression and/or aberrant activity of a cathepsin L-like protease.

Also provided is the use of cathepsin propeptide or a nucleic acid encoding a cathepsin propeptide in the preparation of a medicament for the treatment of a condition associated with overexpression and/or aberrant activity of a cathepsin L-like protease.

In a further aspect, the invention provides a pharmaceutical composition comprising a cathepsin propeptide or a nucleic acid encoding a cathepsin propeptide.

Cathepsin L-like proteases consist of cathepsin L protease, cathepsin S protease, cathepsin K protease and cathepsin V proteases.

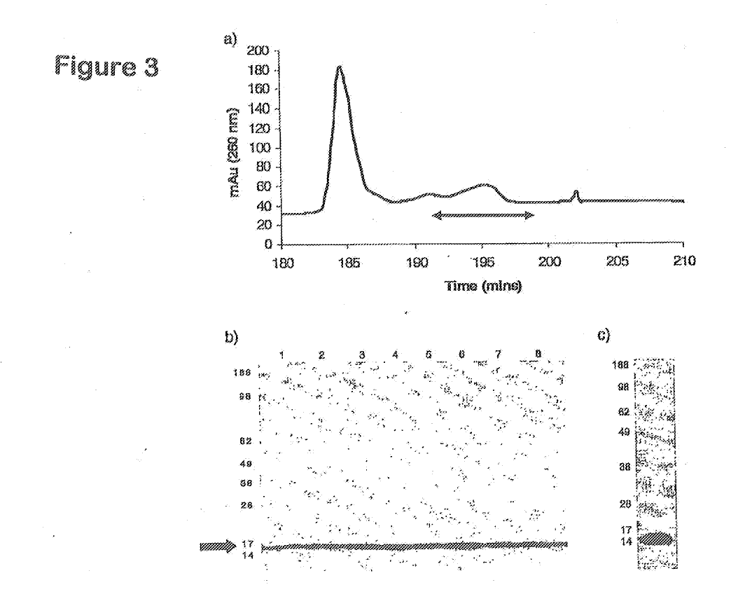

Cathepsin propeptides for use in the invention may be a cathepsin propeptide of any species. In one embodiment, the species is a mammalian species, for example, mouse, rat, human etc. In one embodiment, the cathepsin propeptide is a human cathepsin propeptide, for example the human cathepsin propeptide having amino acid sequence corresponding to amino acid residues 17 to 113 of the cathepsin S protease as disclosed in accession no M90696, (reproduced as amino acid residues 13 to 109 of the amino acid sequence shown in FIG. 3).

In the context of the present invention, cathepsin propeptides include cathepsin propeptides comprising the amino acid sequence of a wild type mammalian cathepsin propeptide or a fragment or derivative thereof. In one embodiment, the cathepsin propeptide consists of the peptide having the amino acid sequence of a wild type mammalian cathepsin propeptide.

In one embodiment, the cathepsin propeptide or derivative or fragment thereof for use in the invention is a cathepsin S propeptide, for example, consisting of amino acids 17 to 113 of the cathepsin S protease as disclosed in accession no M90696 (reproduced as amino acid residues 13 to 109 of the amino acid sequence shown in FIG. 3b).

The cathepsin propeptide may incorporate a tag, for example a polyHis tag. In one embodiment, the cathepsin propeptide is the cathepsin propeptide having a poly His tag as shown as the amino acid sequence 1-118 of FIG. 3b.

As described in the Examples, a particularly potent inhibition of tumour invasion was demonstrated in a tumour invasion assay when using a cathepsin propeptide fused to an antibody Fc portion. Given that by providing the cathepsin propeptide as a fusion peptide with the Fc portion, the shape of the molecule would be expected to change, it was particularly surprising that, not only did the cathepsin propeptide retain its ability to inhibit the invasion but that its inhibitory activity was significantly greater than that of the cathepsin propeptide without the Fc portion.

Accordingly, in one embodiment of the invention, the cathepsin propeptide comprises an antibody Fc portion. In one such embodiment, the Fc portion is an IgG Type b Fc portion, for example a murine IgG Type b Fc portion.

Cathepsin propeptides for use in the invention may be used in the treatment of any condition with which aberrant expression of a cathepsin L-like-protease is associated. For example, conditions in which the invention may be used include, but are not limited to, neoplastic disease, inflammatory disorders, neurodegenerative disorders, autoimmune disorders, asthma, or atherosclerosis. In one embodiment of the invention, the condition is a condition associated with overexpression and/or aberrant activity of cathepsin S.

Preferred features of each aspect of the invention are as for each of the other aspects mutatis mutandis unless the context demands otherwise.

### DETAILED DESCRIPTION

As described above and demonstrated in the examples, the present inventors have shown that, contrary to expectations, cathepsin propeptides act in tumour invasion assays to potently inhibit the activity of cathepsins L-type proteases, in particular the activity of CatS, CatL, CatV and CatK, and have shown that cathepsin propeptides potently block tumour invasion in breast, colon, prostate and astrocytoma tumour models using a modified Boyden chamber invasion assay. These results demonstrate the effect that this molecule can have on tumorigenesis to attenuate the progression of invasive or metastatic cancer cells.

### Cathepsin Propeptides

Cathepsin propeptides for use in the invention may be a cathepsin propeptide of any species, for example a mammalian species. In one embodiment, the cathepsin propeptide is a human cathepsin propeptide, for example a cathepsin propeptide comprising amino acids having the sequence corresponding to that of amino acid residues 17 to 113 of M90696 (reproduced as amino acid residues 13 to 109 of the amino acid sequence shown in FIG. 3).

In the context of the present invention, cathepsin propeptides include cathepsin propeptides comprising the amino acid sequence of a wild type mammalian cathepsin propeptide or a fragment or derivative thereof. In one embodiment, the cathepsin propeptide consists of the peptide having the amino acid sequence of a wild type mammalian cathepsin propeptide.

In one embodiment, the cathepsin propeptide or derivative or fragment thereof for use in the invention is a cathepsin L-type protease propeptide. For example, the cathepsin propeptide or derivative or fragment thereof for use in the invention may be a cathepsin S propeptide.

A fragment of a cathepsin propeptide for use in the invention generally means a stretch of amino acid residues; of at least 10 contiguous amino acids, typically at least 20, for example at least at least 30, such as at least 50 or more consecutive amino acids of a wild-type cathepsin propeptide.

A “derivative” of cathepsin propeptide for use in the invention typically means a polypeptide which, compared with a wild-type cathepsin propeptide, is modified by varying the amino acid sequence, e.g. by manipulation of the nucleic acid encoding the protein or by altering the protein itself. Such derivatives may involve insertion, addition, deletion and/or substitution of one or more amino acids. In one embodiment, derivatives may involve the insertion, addition, deletion and/or substitution of 25 or fewer amino acids, for example 15 or fewer, typically 10 or fewer, such as 5 or fewer for example of 1 or 2 amino acids only. Derivatives of the cathepsin propeptide peptide may contain other amino acids than the natural amino acids or substituted amino acids. For example, derivatives can be obtained from peptidomimetics.

In one embodiment of the invention, the cathepsin propeptide comprises an Fc portion.

Fragments or derivatives of cathepsin propeptides which may be used in the invention preferably retain cathepsin propeptide functional activity, said activity being the ability to inhibit tumour invasion, for example, in a tumour model, for example using a modified Boyden chamber invasion assay. In one embodiment, the cathepsin propeptide fragments or derivatives retain at least 50%, for example at least 75%, at least: 85%, or at least 90% of the tumour invasion inhibition activity of the wild-type human cathepsin propeptide.

Cathepsin propeptides, fragments and derivatives for use in the invention may be produced using any method known in the art.

However, the present inventors have developed a novel simplified method for the simplified recombinant production of cathepsin propeptide. As shown in the Examples, the inventors have demonstrated that recombinant cathepsin propeptides may be successfully expressed with an N-terminal hexahistidine tag and purified using refold metal ion affinity chromatography (IMAC).

Accordingly, in one aspect of the invention, the cathepsin propeptide is produced by a method involving a purification step involving metal ion affinity chromatography (IMAC).

Indeed, in a further independent aspect of the invention, there is provided a method for the recombinant production of cathepsin propeptides, said method comprising expressing a cathepsin propeptide with an N-terminal polyhistidine tag and purifying the expressed propeptide using metal ion affinity chromatography (MAG). In one embodiment, the propeptide is purified in the presence of urea containing buffer.

The principles of IMAC are generally appreciated by those of skill in the art. It is believed that adsorption is predicated on the formation of a metal coordination complex between a metal ion, immobilized by chelation on the adsorbent matrix, and accessible electron donor amino acids on the surface of the protein to be bound.

Similarly, the addition of poly-histidine tags to recombinant proteins is well known in the art (for example, see U.S. Pat. No. 4,569,794.

### Nucleic Acid

Nucleic acid of and for use in the present invention may comprise DNA or RNA. It may be produced recombinantly, synthetically, or by any means available; to those in the art, including cloning using standard techniques.

The nucleic acid may be inserted into any appropriate vector. In one embodiment the vector is an expression vector and the nucleic acid is operably linked to a control sequence which is capable of providing expression of the nucelic acid in a host cell. A variety of vectors may be used. For example, suitable vectors may include viruses (e.g. vaccinia virus, adenovirus, baculovirus etc); yeast vectors, phage, chromosomes, artificial chromosomes, plasmids, or cosmid DNA.

The vectors may be used to introduce the nucleic acids into a host cell. A wide variety of host cells may be used for expression of the nucleic acid for use in the invention. Suitable host cells for use in the invention may be prokaryotic or eukaryotic. They include bacteria, e.g. E. coli, yeast, insect cells and mammalian cells. Mammalian cell lines which may be used include Chinese hamster ovary cells, baby hamster kidney cells, NSO mouse melanoma cells, monkey and human cell lines and derivatives thereof and many others.

A host cell strain that modulates the expression of, modifies, and/or specifically processes the gene product may be used. Such processing may involve glycosylation, ubiquination, disulfide bond formation and general post-translational modification.

For further details relating to known techniques and protocols for manipulation of nucleic acid, for example, in preparation of nucleic acid constructs, mutagenesis, sequencing, introduction of DNA into cells and gene expression, and analysis of proteins, see, for example, Current Protocols in Molecular Biology, 2nd ed., Ausubel et al. eds., John Wiley & Sons, 1992 and, Molecular Cloning: a Laboratory Manual: 3^{rd} edition Sambrook et al., Cold Spring Harbor Laboratory Press, 2000.

### Treatment

“Treatment” includes any regime that can benefit a human Or non-human animal. The treatment may be in respect of an existing condition or may be prophylactic (preventative treatment). Treatment may include curative, alleviation or prophylactic effects.

The cathepsin propeptides, nucleic acids and methods of and for use in the invention may be used in the treatment of a number of medical conditions. These include inflammatory disorders neurodegenerative disorders, autoimmune diseases, cancer, asthma and atherosclerosis. In particular, they may be used in the treatment of conditions associated with overexpression (i.e. greater than in similar comparable normal healthy cells) and/or aberrant activity (eg greater than in similar comparable normal healthy cells) of cathepsin proteases.

The propeptides, nucleic acids and methods of and for use in the invention may be used in the treatment of cancers. “Treatment of cancer” includes treatment of conditions caused by cancerous growth and includes the treatment of neoplastic growths or tumours. The invention may be particularly useful in the treatment of existing cancer and in the prevention of the recurrence of cancer after initial treatment or surgery.

Examples of tumours that can be treated using the invention include, for instance, sarcomas, including osteogenic and soft tissue sarcomas, carcinomas, e.g., breast-, lung-, bladder-, thyroid-, prostate-, colon-, rectum-, pancreas, stomach-, liver-, uterine-, prostate, cervical and ovarian carcinoma, lymphomas, including Hodgkin and non-Hodgkin lymphomas, neuroblastoma, melanoma, myeloma, Wilms tumor, and leukemias, including acute lymphoblastic leukaemia and acute myeloblastic leukaemia, astrocytomas, gliomas and retinoblastomas.

In one embodiment, the cancer is selected from breast cancer, colon cancer, prostate cancer and astrocytomas.

Inflammatory and/or autoimmune disorders which may be treated using the invention include multiple sclerosis, Grave's Disease, inflammatory muscle disease and rheumatoid arthritis.

Neurodegenerative disorders which may be treated using the binding members, nucleic acids and methods of the invention include, but are not limited to, Alzheimer's Disease, Parkinson's Disease, Multiple Sclerosis and Creutzfeldt-Jakob disease.

Other conditions which may be treated using the methods of the invention include atherosclerosis and tuberculosis. Evidence has been shown linking atherosclerosis and obesity with aberrant CatS. Cathepsin L has been shown to process TB antigens in infections, thus perhaps preventing their proper processing.

### Pharmaceutical Compositions

The propeptides and nucleic acids of and for use in the invention may be administered as a pharmaceutical composition. Pharmaceutical compositions according to the present invention, and for use in accordance with the present invention may comprise, in addition to active ingredients, a pharmaceutically acceptable excipient, a carrier, buffer stabiliser or other materials well known to those skilled in the art (see, for example, Remington: The Science and Practice of Pharmacy, 21st edition, Gennaro A R, et al, eds., Lippincott Williams & Wilkins, 2005). Such materials may include buffers such as acetate, Tris, phosphate, citrate, and other organic acids; antioxidants; preservatives; proteins, such as serum albumin, gelatin, or immunoglobulins; hydrophilic polymers such aspolyvinylpyrrolidone; amino acids such as glycine, glutamine, asparagine, histidine, arginine, or lysine; carbohydrates; chelating agents; tonicifiers; or surfactants.

The composition may also contain one or more further active compounds selected as necessary for the particular indication being treated, preferably with complementary activities that do not adversely affect the activity of the propeptide, nucleic acid or composition of the invention. For example, in the treatment of cancer, in addition to an a cathepsin propeptide, the formulation may comprise an antibody which binds one or more cathepsin L-type proteases, or an antibody to some other target such as a, growth factor that e.g. affects the growth of the particular cancer, and/or a chemotherapeutic agent.

The active ingredients (e.g. propeptides and/or chemotherapeutic agents) may be administered via microspheres, microcapsules liposomes, other microparticulate delivery systems. For example, active ingredients may be entrapped within microcapsules which may be prepared, for example, by coacervation techniques of by interfacial polymerization, for example, hydroxymethylcellulose or gelatinmicrocapsules and poly-(methylmethacylate) microcapsules, respectively, in colloidal drug delivery systems (for example, liposomes, albumin microspheres, microemulsions, nano-particles and nanocapsules) or in macroemulsions. For further details, see Remington: The Science and Practice of Pharmacy, 21st edition, Gennaro A R, et al, eds,. Lippincott Williams & Wilkins, 2005.

Sustained-release preparations may be used for delivery of active agents. Suitable examples of sustained-release preparations include semi-permeable matrices of solid hydrophobic polymers containing the antibody, which matrices are in the form of shaped articles, e.g. films, suppositories or microcapsules. Examples of sustained-release matrices include, polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L glutamate, non-degradable ethylene-vinyl acetate, degradable lactic acid-glycolic acid copolymers, and poly-D-(−)-3-hydroxybutyric acid.

The propeptides described herein are intended, at least in some embodiments, to be administered to a human or other mammal for medical treatment.

Peptides are typically administered parenterally, and may be readily metabolized by plasma proteases. Oral administration, which is perhaps the most attractive route of administration, may be even more problematic. In the stomach, acid degrades and enzymes break down peptides. Those peptides that survive to enter the intestinal intact are subjected to additional proteolysis as they are continuously barraged by a variety of enzymes, including gastric and pancreatic enzymes, exo- and endopeptidases, and brush border peptidases. As a result, passage of peptides from the lumen of the intestine into the bloodstream can be severely limited. However, various prodrugs have been developed that enable parenteral and oral administration of therapeutic peptides.

Peptides can be conjugated to various moieties, such as polymeric moieties, to modify the physiochemical properties of the peptide drugs, for example, to increase resistance to acidic and enzymatic degradation and to enhance penetration of such drugs across mucosal membranes. For example, Abuchowski and Davis have described various methods for derivatizating enzymes to provide water-soluble, non-immunogenic, in vivo stabilized products (“Soluble polymers-Enzyme adducts,” Enzymes as Drugs, Eds. Holcenberg and Roberts, J. Wiley and Sons, New York, N.Y. (1981)). Abuchowski and Davis discuss various ways of conjugating enzymes with polymeric materials, such as dextrans, polyvinyl pyrrolidones, glycopeptides, polyethylene glycol and polyamino acids. The resulting conjugated polypeptides retain their biological activities and solubility in water for parenteral applications. U.S. Pat. No. 4,179,337 teaches coupling peptides to polyethylene glycol or polypropropylene glycol having a molecular weight of 500 to 20,000 Daltons to provide a physiologically active non-immunogenic water soluble polypeptide composition. The polyethylene glycol or polypropylene glycol protects the polypeptide from loss of activity and the composition can be injected into the mammalian circulatory system with substantially no immunogenic response.

U.S. Pat. No. 5,681,811, U.S. Pat. No. 5,438,040 and U.S. Pat. No. 5,359,030 disclose stabilized, conjugated polypeptide complexes including a therapeutic agent coupled to an oligomer that includes lipophilic and hydrophilic moieties. Garmen, et al. describe a protein-PEG prodrug (Garman, A J., and Kalindjian, S. B., FEBS Lett., 1987, 223, 361-365). A prodrug can be prepared using this chemistry, by first preparing a maleic anhydride reagent from polydispersed MPEG5000 and then conjugating this reagent to the peptides disclosed herein. The reaction of amino acids with maleic anhydrides is well known. The hydrolysis of the maleyl-amide bond to reform the amine-containing drug is aided by the presence of the neighboring free carboxyl group and the geometry of attack set up by the double bond. The peptides can be released (by hydrolysis of the prodrugs) under physiological conditions.

Such strategies may be employed to deliver the propeptides for use in the present invention.

The peptides can also be coupled to polymers, such as polydispersed PEG, via a degradable linkage, for example, the degradable linkage shown (with respect to pegylated interferon α-2b) in Roberts, M. J., et al., Adv. Drug Delivery Rev., 2002, 54, 459-476.

The peptides can also be linked to polymers such as PEG using 1, 6 or 1,4 benzyl elimination (BE) strategies (see, for example, Lee, S., et al., Bioconjugate Chem., (2001), 12, 163-169; Greenwald, R. B., et al., U.S. Pat. No. 6,180,095, 2001; Greenwald, R. B., et al., J. Med. Chem., 1999, 42, 3657-3667.); the use of trimethyl lock lactonization (TML) (Greenwald, R. B, et al., J. Med. Chem., 2000, 43, 475-487); the coupling of PEG carboxylic acid to a hydroxy-terminated carboxylic acid linker (Roberts; M. J., J. Pharm. Sci., 1998, 87(11), 1440-1445), and PEG prodrugs involving families of MPEG phenyl ethers and MPEG benzamides linked to an amine-containing drug via m aryl carbamate (Roberts, M. L, et al., Adv. Drug Delivery Rev., 2002, 54, 459-476), including a prodrug structure involving a meta relationship between the carbamate and the PEG amide or ether (U.S. Pat. No. 6,413,507); and prodrugs involving a reduction mechanism as opposed to a hydrolysis mechanism (Zalipsky, S., et al., Bioconjugate Chem., 1999, 10(5), 703-707).

Some approaches involve using enzyme inhibitors to slow the rate of degradation of proteins and peptides in the gastrointestinal tract and may be used for the propeptides described herein; manipulating pH to inactivate local digestive enzymes; using permeation enhancers to improve the absorption of peptides by increasing their paracellular and transcellular transports; using nanoparticles as particulate carriers to facilitate intact absorption by the intestinal epithelium, especially, Peyer's patches, and to increase resistance to enzyme degradation; liquid emulsions to protect the drug from chemical and enzymatic breakdown in the intestinal lumen; and micelle formulations for poorly water-solubulized drugs.

In some cases, the peptides can be provided in a suitable capsule or tablet with an enteric coating, so that, the peptide is not released in the stomach. Alternatively, of additionally, the peptide can be provided as a prodrug. In one embodiment, the peptides are present in these drug delivery devices as prodrugs.

Free amino, hydroxyl, or carboxylic acid groups of the peptides can be used to convert the peptides into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of various polymers, for example, polyalkylene glycols such as polyethylene glycol. Prodrugs also include compounds wherein carbonates, carbamates, amides and alkyl esters are covalently bonded to the above peptides through the C-terminal carboxylic acids.

Prodrugs comprising the peptides (propeptides) of the invention or pro-drugs from which peptides of the invention (including analogues and fragments) are released of are releasable are considered to be derivatives of the invention.

### Peptidomimetics

The present invention further encompasses the use of mimetic propeptides which can be used as therapeutic peptides. Mimetic pro peptides are short, peptides which mimic the biological, activity of the cathepsin propeptides described herein. Such mimetic, peptides can be obtained from methods known in the art such as, but not limited to, phage display of combinatorial chemistry. For example, the method disclosed by Wrighton, et al., Science 273:458-463 (1996) can be used to generate mimetic QUB 698.8 peptides.

As described above nucleic acids encoding cathepsin propeptides may also be used in methods of treatment. Such nucleic acids; may be delivered to cells of interest using any suitable technique known in the art. Nucleic acid (optionally contained in a Vector) may be delivered to a patient's cells using in vivo or ex vivo techniques. For in vivo techniques, transfection with viral vectors (such as adenovirus, Herpes simplex I virus, or adeno-associated virus) and lipid-based systems (useful lipids for lipid-mediated transfer of the gene are DOTMA, DOPE and DC-Choi, for example) may be used (see for example, Anderson et al., Science 256:808-813 (1992). See also WO 93/25673).

In ex vivo techniques, the nucleic acid is introduced into isolated cells of the patient with the modified cells being administered to the patient either directly or, for example, encapsulated within porous membranes which are implanted into the patient (see, e.g. U.S. Pat. Nos. 4,892,538 and 5,283,187). Techniques available for introducing nucleic acids into viable cells may include the use of retroviral vectors, liposomes, electroporation, microinjection, cell fusion, DEAE-dextran, the calcium phosphate precipitation method, etc.

The propeptide, nucleic acid, agent, product or composition may be administered in a localised manner to a tumour site or other desired site or may be delivered in a manner in which it targets tumour or other cells. Targeting therapies may be used, to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

### Dose

The propeptides, nucleic acids or compositions of the invention are preferably administered to an individual in a “therapeutically effective amount”, this being sufficient to show benefit to the individual. The actual dosage regimen will depend on a number of factors including the condition being treated, its severity, the patient being treated, the agent being used, and will be at the discretion of the physician.

The optimal dose can be determined by physicians based on a number of parameters including, for example, age, sex, weight, severity of the condition being treated, the active ingredient being administered and the route of administration.

The invention will now be described further in the following non-limiting examples. Reference is made to the accompanying drawings in which:

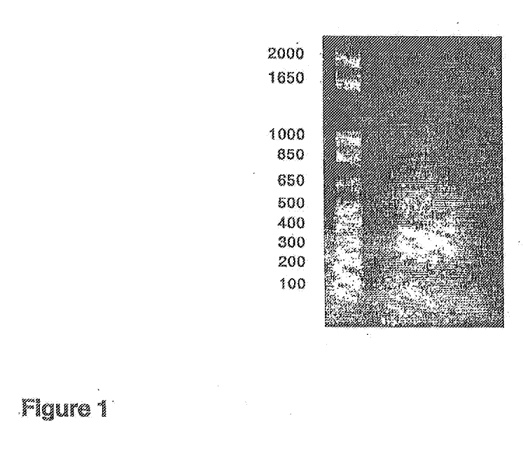

FIG. 1 illustrates the amplification of CatSPP. The cDNA sequence of the CatSPP was amplified from a human spleen cDNA library. A single band of approximately 330 bp was produced, equivalent in size to that expected for the CatSPP cDNA sequence.

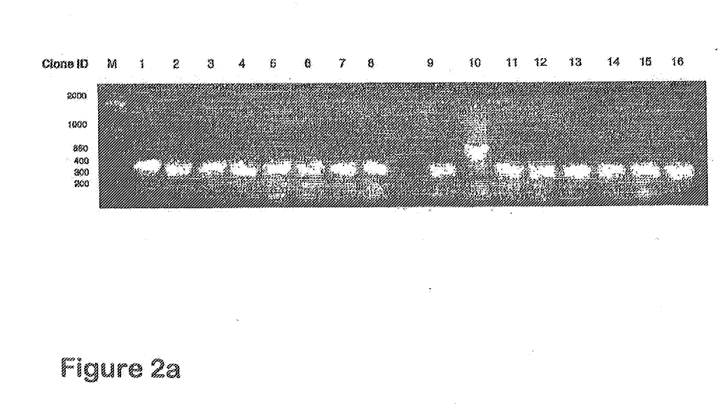

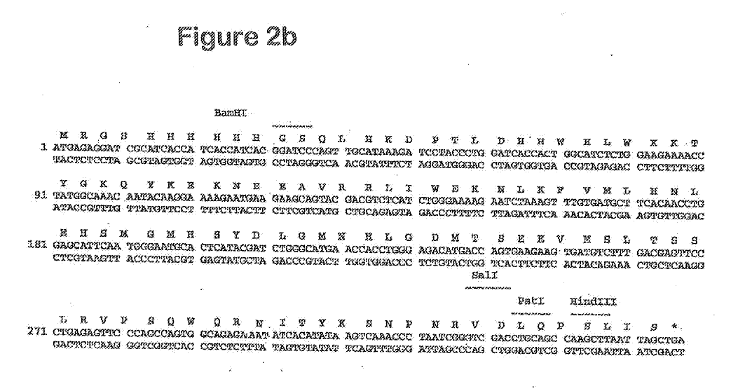

FIG. 2a illustrates the results of Colony PCR from CatS PP cloning into pQE-30

FIG. 2b illustrates the DNA and protein sequence for the complete reading frame of the rCatSPP is shown as a result of its insertion into pQE30.

FIG. 3 illustrates Purification of the rCatSPP protein: a) an elution profile of the rCatSPP b) shows SDS-PAGE analysis of fractions from the second broad peak, as indicated by the arrow c) Immunoblot of purification fractions using ah anti-polyhistidine tag antibody.

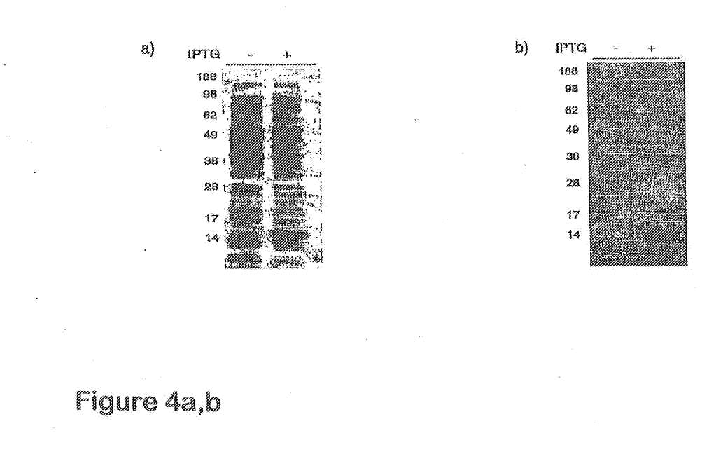

FIG. 4 illustrates the inducibility of rCatSPP expression by IPTG as demonstrated by SDS-PAGE and western blotting, a) Analysis of bacterial lysates by SDS-PAGE and coomassie blue staining b) Blotting with an anti-polyhistidine tag antibody. Molecular weight markers are indicated at the left of each image (kDa).

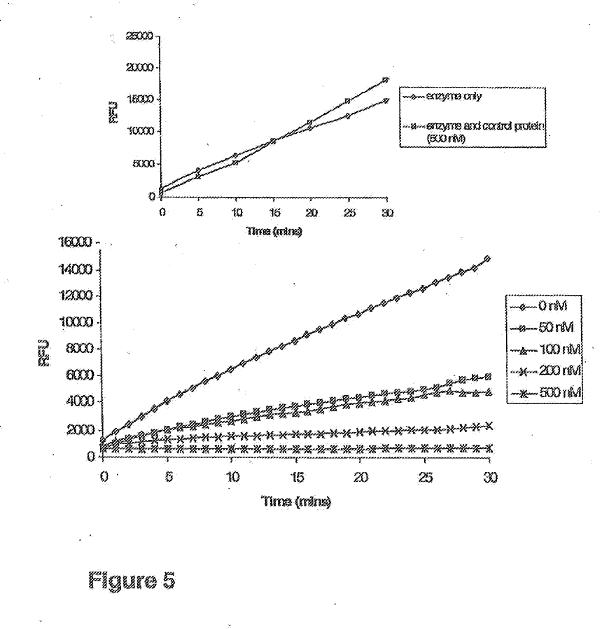

FIG. 5 illustrates progress curves for the hydrolysis of Cbz-Val-Val-Arg-AMC in the presence of rCatSPP. A control (His)6-tagged protein, produced from the same vector and purified in the same manner, was used as a control (500 nM) (inset).

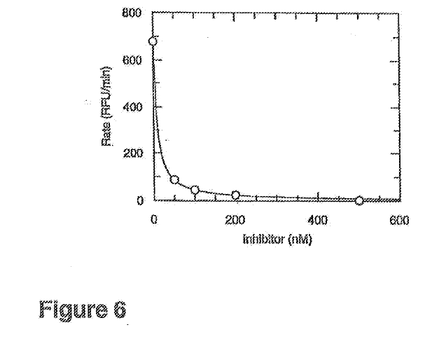

FIG. 6 shows a graph of non-linear regression analysis (Morrison and Walsh, 1988) allowing for the determination of the inhibition constant (Ki).

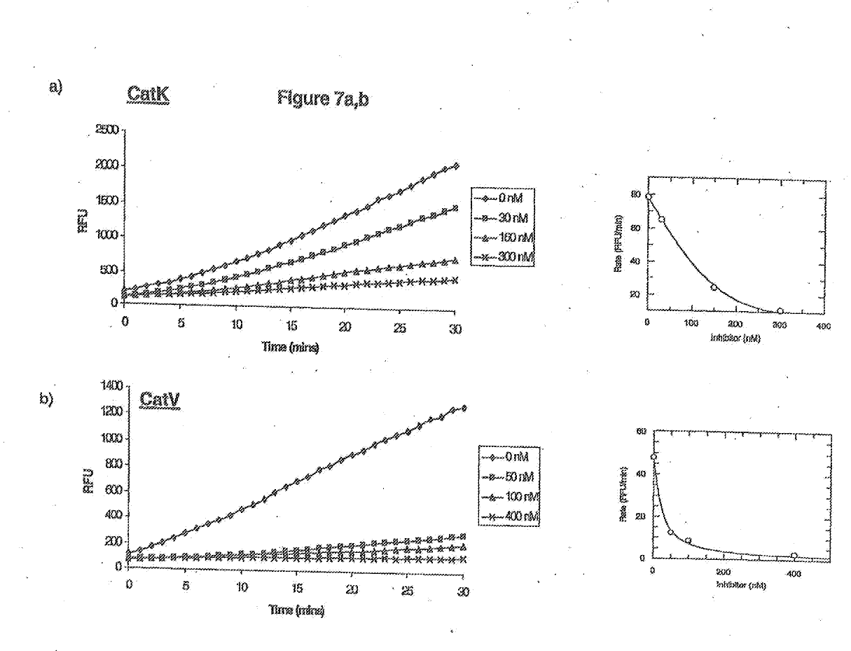

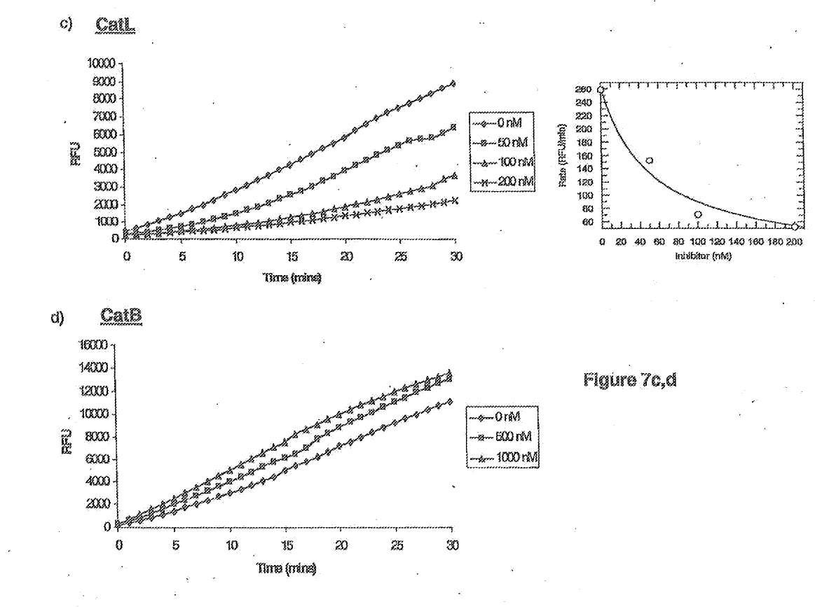

FIG. 7: Inhibition of cathepsins K, V, L and B by the rCatSPP protein using fluorometric assays.

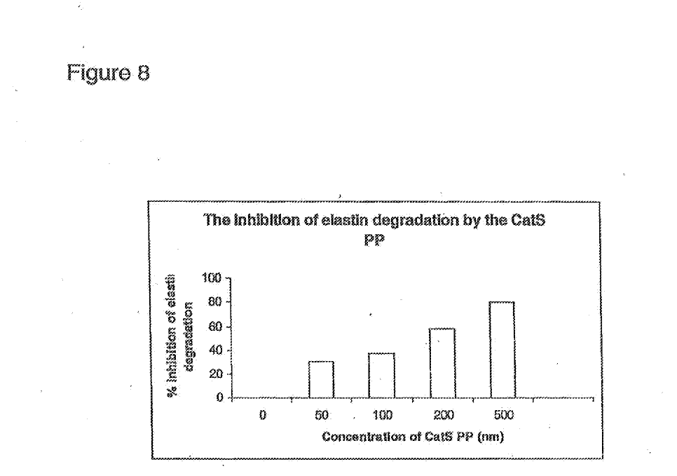

FIG. 8 illustrates inhibition of CatS elastinolytic activity. The fluorogenic substrate Elastin-DQ was used to monitor the elastinolytic turnover of CatS in the presence of CatSPP (50-500 nM) over a 60 minute incubation

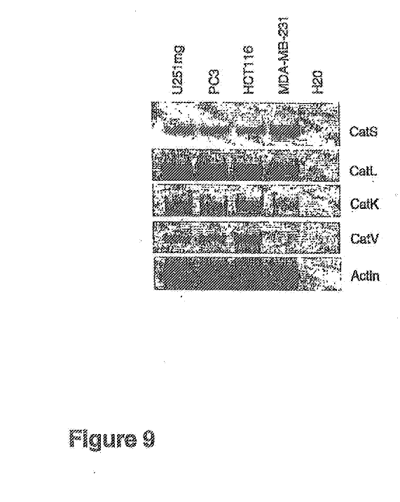

FIG. 9 shows relative expression of CatL-like proteases in malignant cell lines.

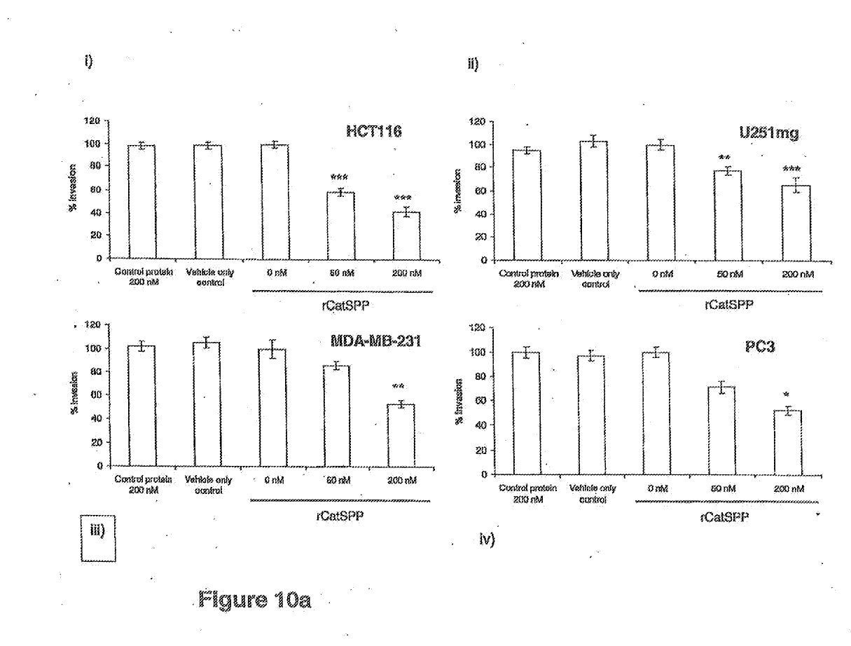

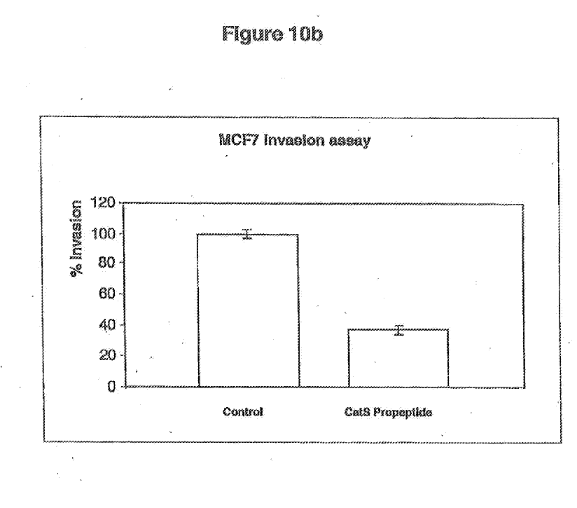

FIG. 10a shows the results of in vitro invasion assays in four human malignant cell lines by; i-iv: HCT116, U251 mg, MDA-MB-231 and PC3.

FIG. 10b shows the results of in vitro invasion assays in MCF-7 cells.

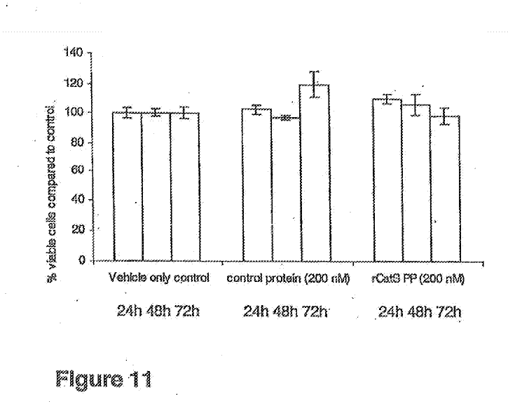

FIG. 11 shows the results of an MTT assay assessing cytotoxic or proliferative effects of the rCatSPP protein.

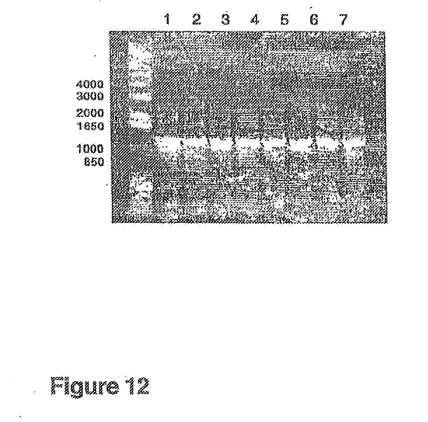

FIG. 12 illustrates Colony PCR analysis of CatSPP cloning into pRSET A-Fc.

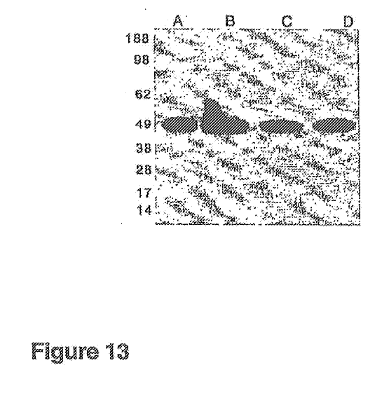

FIG. 13 shows the expression of the rCatSPP-Fc from the pRSET A vector was induced by the addition of IPTG as resolved by SDS-PAGE and western blotting performed using an anti-polyhistidine tag antibody.

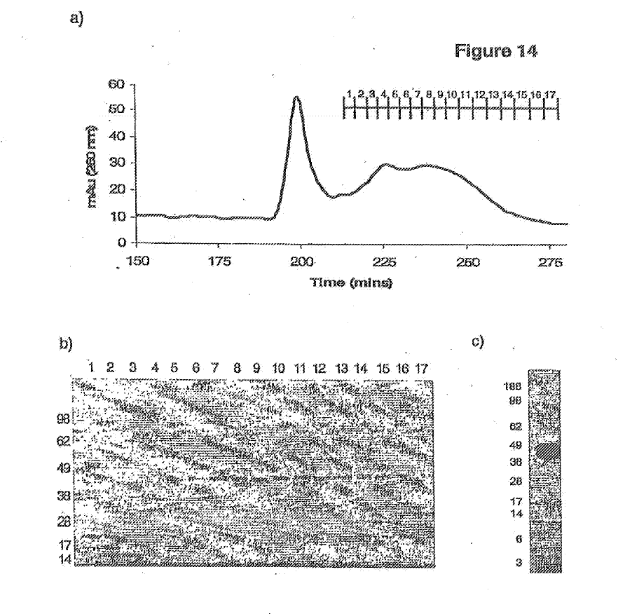

FIG. 14: Purification of rCatSPP-Fc.
a) shows the purification profile shows two distinct peaks, a sharp peak at after approximately 200 mins and a second broader peak between 225 and 250 mins.
b) shows the analysis of eluted fractions from the purification.
c) shows analysis of purification fractions by western blotting using an anti-polyhistidine tag monoclonal antibody.

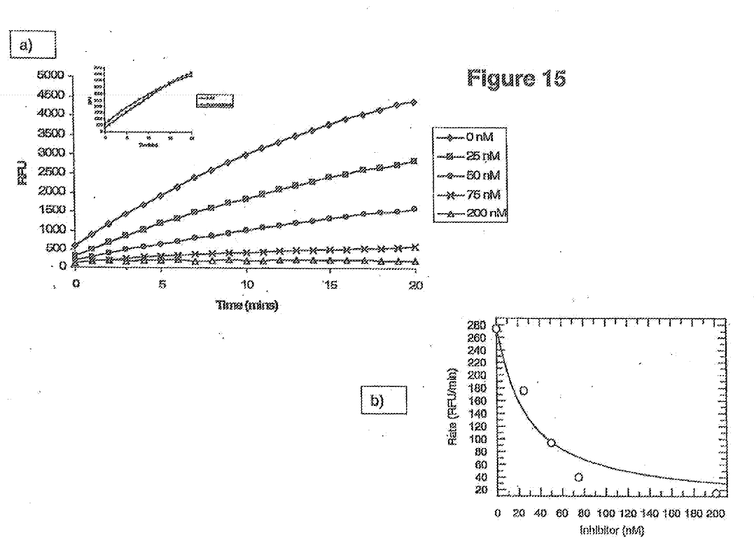

FIG. 15 illustrates CatS inhibition by the rCatSPP-Fc using a fluorometric assay.

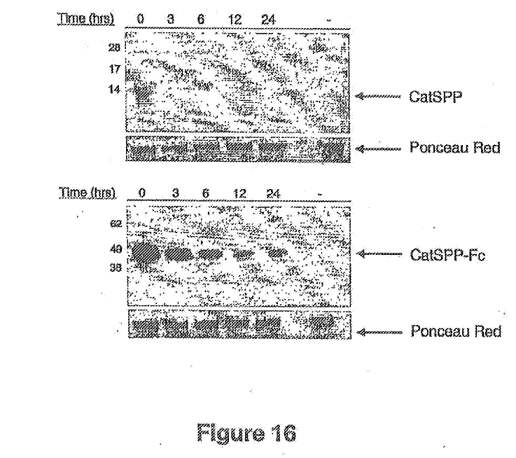

FIG. 16 illustrates Western blots demonstrating the stability of CatS PP versus CatS PP-Fc.

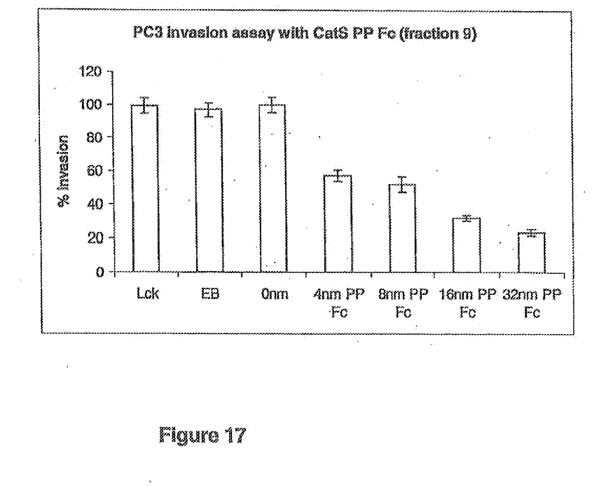

FIG. 17 illustrates a histogram showing quantitative summary of the PC3 invasion assay in the presence of is CatSPP Fc

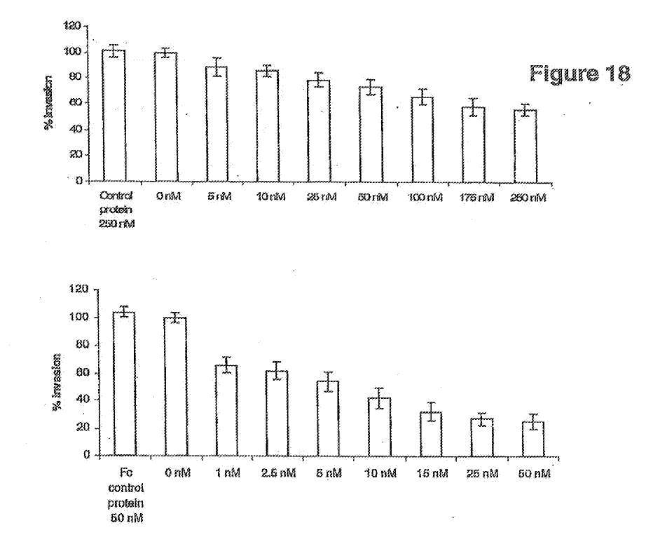

FIG. 18 illustrates histograms showing quantitative summary of the HCT116 invasion assay in the presence of CatSPP and CatSPP-Fc recombinant proteins.

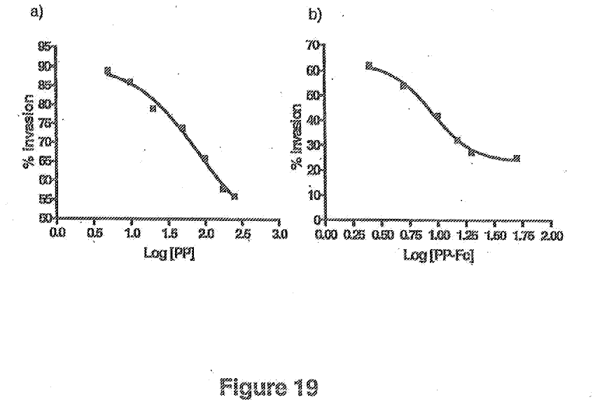

FIG. 19 shows dose-response curves used to determine EC50 values for rCatS PP and rCatS PP-Fc in MDA-MB-231 tumour cells.

### EXAMPLES

### Materials and Methods

### Cloning and Expression of CatSPP

The human CatSPP, residues 17-113, was amplified from a human spleen cDNA library (Origene) using primers CATSPPF (5′ TTT TTTGGATCCCAGTTGCATAAAGATCCTAC) and CATSPPR (5′ TTTTTTGTCGACCCGATTAGGGTTTGA) containing BamHI and Sail restriction sites respectively (as underlined). The expected band of 330 bp was visualised by agarose electrophoresis. This band was gel purified and cloned using BamHI and SalI into pQE30 (Qiagen), which incorporated ah N terminal hexahistdine tag for downstream manipulations; Positive clones were identified by colony PCR and sequence aligned to accession number M90696. A single verified clone was used in subsequent experiments.

### Cloning and Expression of CatSPP-Fc

For cloning of the CatSPP into the pRSET-Fc vector, the DNA sequence was amplified using primers CATSPPFCF (5′ TTTTTTGGATCCCAGTTGCATAAA GAT) and CATSPPFCR (5′ TTTTTTGTCGACTATCCGATTAGGGTT), again with BamHI and SalI restriction enzyme sites respectively (as underlined). The amplified band was gel excised and cloned into the pRSET bacterial expression vector which had previously been engineered to contain an IgG₂ Fc domain. Positive clones were identified by colony PCR and sequence aligned to accession number M90696. A single verified clone was used in all subsequent experiments.

### Protein Expression and Purification of CatSPP and CatSPP-Fc

For expression analysis, the CatSPP positive clone was transformed into TOP10F′ cells and cultured in shaker flasks (500 ml) until reaching mid log phase (A₅₅₀ 0.5, 37° C.). Expression analysis of the CatSPP-Fc positive clone was performed by transformation using the BL21 (DE3) pLysS strain of E. coli. Expression of both recombinant proteins was induced by the addition of isopropyl-β-D-thiogalactoside (PTG, 1 mM) to the bacterial cultures and propagated for a further 4 hours prior to harvesting. Cell pellets were resuspended and lysed in 50 mM NaH₂PO₄ pH 8.0, containing 8M urea, 300 mM NaCl and 10 mM imidazole. The crude denatured lysate was clarified by centrifugation (10,000 g, 60 minutes at 4° C.), prior to application to a MAC column charged with Ni²⁺ ions (HiTrap 1 ml column, GE Healthcare). Non-specifically bound material was washed from the column using 50 mM NaH₂PO₄ pH 8.0, containing 8 M urea, 300 mM NaCl and 20 mM imidazole, prior to on-column refolding by reduction of the urea from 8 to 0 M over 200 column volumes. Refolded column bound material was washed with a further 20 column volumes of 50 mM NaH₂PO₄ pH 8.0, 300 mM NaCl and 20 mM imidazole, prior to elution with 50 mM NaH₂PO₄ pH 8.0, 300 mM NaCl and 250 mM imidazole. Protein fractions were collected, desalted into PBS and analysed by SDS-PAGE and western blotting to determine purity and integrity. Stocks of purified recombinant protein were stored at −20° C. prior to use.

Inhibition of Cysteine Cathepsins with rCatSPP

Enzymatic assays were used to ascertain the ability of the rCatSPP to inhibit the peptidolytic activity of human cathepsins S, L, K, V and B (Calbiochem). Assays were performed in triplicate in 96-well microtitre plates in the presence of 100 mM sodium acetate, 1 mM ethylenediaminetetraacetate (EDTA), 0.1% Brij and 1 mM: dithiothreitol (DTT) at pH 5.5. CatS activity was monitored using the fluorigenic substrate carbobenzloxy-L-valinyl-L-vahnyl-L-arginylamido-4-methyl coumarin (Z-Val-Val-Arg-AMC, 25 μM), assays for cathepsins L, K and V were performed using carbobenzloxy-L-phenylalanyl-L-arginylamido-4-methyl coumarin (Z-Phe-Arg-AMC, 25 μM) and assays for CatB were performed using carbobenzloxy-L-arginylamido -L-arginylamido-4-methyl coumarin (Z-Arg-Arg-AMG, 25 μM) as substrates. Purified rCatSPP was added to assays as required at various concentrations (0-1000 nM). All experiments were performed using a Cytofluor® 4000 spectrofluorimeter with excitation at 395 nm and emission at 460 nm. To confirm that the rCatSPP-Fc also had the ability to inhibit the activity of CatS, flurometric assays were performed using CatS, Z-Val-Val-Arg-AMC, 25 μM) in the presence of the rCatSPP-Fc (0 nM-200 nM).

### RT-PCR Analysis of Cysteine Cathepsin Expression

The relative expression levels of the cysteine cathepsins S, L, K and V in a panel of human malignant: cell lines was determined by RT-PCR analysis. RNA was extracted from U251 mg, MDA-MB-231, HCT116 and PC3 cell lines using the Absolutely RNA™ RT-PCR Miniprep kit, and quantified using, a spectrophotometer. RT-PCR was performed using the One-Step RT-PCR kit under the following conditions: 50° C. for 30 min, 95° C. for 15 min, and 35 cycles of 94° C. for 1 min, 55° C. for 1 min and 72° C. for 1 min 30 sec, followed by 72° C. for 10 min or as detailed in the text. Amplification of a series of cysteine cathepsins; was performed using the primers detailed in the table below. Amplification of the β-actin gene was used as an internal control to demonstrate equal loading. RT-PCR products were analysed by agarose gel electrophoresis and images were taken under UV light using Kodak ID 3.4 USB software and a digital camera.

### Gene RT-PCR Primer Sequence

| | |
|---|---|
| CatS (F) | GGG TAC CTC ATG TGA CAA G |
| CatS (R) | TCA CTT CTT CAC TGG TCA TG |
| CatL (F) | ATG AAT CCT ACA CTC ATC CTT GC |
| CatL (R) | TCA CAC AGT GGG GTA GCT GGC TGC TG |
| CatK (F) | ATG TGG GGG CTC AAG GTT CTG C |
| CatK (R) | TCA CAT CTT GGG GAA GCT GGC C |
| CatV (F) | ATG AAT CTT TCG CTC GTC CTG GC |
| CatV (R) | TCA CAC ATT GGG GTA GCT GGC |
| Actin (F) | ATC TGG CAC CAC ACC TTC TAC AAT GAG CTG |
| | CG |
| Actin (R) | CGT CAT ACT CCT GCT TGC TGA TCC ACA TCT |
| | GC |

### In-Vitro Invasion Assays

In-vitro invasion assays were performed using a modified Boyden chamber with 12-μm pore membranes (Costar Transwell plates, Corning Costar Corp., Cambridge, Mass., USA). The membranes were coated with Matrigel (100 μg/cm²) (Becton Dickinson, Oxford, UK) and allowed to dry overnight in a laminar flow hood. Cells were added to each well in 500 μl of serum-free medium in the presence of predetermined concentrations of the rCatSPP. All assays were carried out in triplicate and invasion plates were incubated at 37° C. and 5% CO₂ for 24 hours after which cells remaining on the upper surface of the membrane were removed and invaded cells fixed in Carnoy's fixative for 15 minutes. After drying, the nuclei of the invaded cells were stained with Hoechst 33258 (50 ng/ml) in PBS for 30 minutes at room temperature. The chamber insert was washed twice in PBS, mounted in Citifluor and invaded cells were viewed with a Nikon Eclipse TE300 fluorescent microscope. Ten digital images of representative fields from each of the triplicate membranes were taken using a Nikon DXM1200 digital camera at magnification of ×20. The results were analysed using Lucia GF 4.60 by Laboratory Imaging and were expressed as a percentage of invaded cells.

### Cell Viability Assay

Cytotoxic or proliferative effects of the rCatSPP was determined by MTT assay using the HCT116 colorectal carcinoma cell line. Cells were added at a concentration of 1×10⁴ cells per 200 μl to a 96-well plate; 200 nM rCatSPP, a control protein generated from the same vector under identical conditions and a vehicle only control were added to the cells and incubated for 24, 48 and 72 hrs at 37° C. and 5% CO2. After this the medium was carefully removed and 200 μl of 0.5 mg/ml 3-4,5-dimethylthiazol-2,5 diphenyl tetrazolium bromide (MTT) was added and incubated at 37° C. for 2 hr. The MTT reagent was removed and the insoluble formazan crystals were dissolved in 100 μl of DMSO. Absorbance was measured at 570 nm and the results were expressed as a percentage of cell viability or proliferation relative to each vehicle-only control. All tests were performed in quintuplicate.

### Results and Discussion

Previously the purification of the CatSPP has been achieved by a number of different approaches. Maubach and co-workers produced CatSPP from an Escherichia coli expression system, isolating the peptide, corresponding to residues 16-114, from inclusion bodies by refolding against a GdnHCl concentration gradient (Maubach et al., 1997). Guay and colleagues also produced the PP (residues 17-114) in E. coli, using the alternative approach of producing it as a glutathione S-transferase (GST) G-terminal fusion. The recombinant protein was again produced in inclusion bodies, which were subsequently refolded against a GdnHCl concentration gradient, prior to affinity purification on a GST-Sepharose column and the PP removed from the GST fusion by a thrombin cleavage step (Guay et al., 2000). This latter procedure has also been used for the production of the CatKPP and CatLPP. Both these previous methods produced bioactive protein, but are laborious and time consuming, particularly with the isolation and refolding of inclusion bodies. In an effort to determine a more rapid simplified method for the production of CatSPP, the inventors expressed the peptide (residues 17-113) with an N terminal hexahistidine tag and purified the protein by refold MAC.

Using the gene specific primers CATSPPF and CATSPPR detailed, the open reading frame encoding the propeptide region (residues 17-113) were amplified from a commercially available cDNA library by polymerase chain reaction (PCR). When analysed by agarose electrophoresis, a band of the expected size was visualised (FIG. 1). Following gel extraction, the band was cloned into a commercially available vector (pQE30). The analysis of 16 clones by colony PCR reveals a band of approximately 650 bp amplified from colony 10 (FIG. 2a. This would suggest that only colony 10 may contain the CatSPP cDNA sequence cloned successfully into the pQE-30 bacterial expression vector.

DNA sequenced for full validation (sequences aligned to Accession number M90696) (see FIG. 2b). A selected clone was used for all further propagation and fermentation, from which the rCatSPP species was isolated for further study.

rCatSPP expression from the validated clone was then analysed, firstly for over-expression of the protein and verification that it contained an N terminal histag and was the expected molecular weight of 16 kDa and that the expression of the protein was under the control of the T5 promoter, inducible with IPTG:

The rCatSPP was expressed from the pQE-30 bacterial expression vector and purified using on-column refolding MAC. As shown in FIG. 3a) The elution profile of the rCatSPP contains several peaks; a sharp initial peak after 185 mins, followed by a broad peak between 190 and 195 mins. Fractions from the second broad peak, as indicated by the arrow in FIG. 3b, were resolved by SDS-PAGE and revealed the presence of a single highly purified band, with a molecular weight of approximately 16 kDa, corresponding to that predicted for the (His)6-tagged rCatSPP. FIG. 3c) shows immunoblotting of purification fractions using an anti-polyhistidine tag antibody confirm the presence of a his-tagged species at approximately 16 kDa.

The inducibility of rCatSPP expression by IPTG was demonstrated by SDS-PAGE and western blotting (FIG. 4). The analysis of bacterial lysates by SDS-PAGE and coomassie blue staining shows the presence of the rCatSPP at approximately 16 kDa in lane b which has been induced but not in uninduced lane a. (FIG. 4a) The transfer of the bacterial lysates to nitrocellulose membrane and blotting with an anti-polyhistidine tag antibody confirms expression of the protein in the induced lane b only. (FIG. 4b) Molecular weight markers are indicated at the left of each image (kDa).

After final desalting into PBS, the propeptide was then tested for its biological activity. The biological activity of the rCatSPP protein was ascertained by fluorometric assay using CatS and the fluorigenic substrate Cbz-Val-Val-Arg-AMC in the presence of predetermined concentrations of rCatSPP (0 nM to 500 nM). Progress curves for the hydrolysis of Cbz-Val-Val-Arg-AMC in the presence of rCatSPP were plotted and the dose-dependent inhibition of CatS activity was observed (FIG. 5) A control (His)6-tagged protein, produced from the same vector and purified in the same manner, was used as a control (500 nM) to confirm the perturbation of CatS activity was due to the rCatSPP (inset). Assays were all performed in triplicate.

The progress curves are indicative of the action of a slow-binding reversible inhibitor. The apparent first order rate order curves produced were then subjected to non-linear regression analysis (Morrison and Walsh, 1988) where the production of fluorescence [P] over time can be represented by the following equation:

[P]=vₛt−(vₛ−vₒ)(1−exp(−k_{obs}t))/k_{obs}+d (1)

Using GraFit® software, the values for the progression curves shown in FIG. 7a were fitted by non-linear regression analysis into equation (1), producing a graph of vₛ against [I], from which Kᵢ (observed) was determined. This was then corrected to account for competing substrate, as shown in equation (2).

{Kᵢ=K_{i(observed)}/(1+[S]/Kₘ)} (2)

Using this analysis, K; values were calculated for inhibition of CatS with rCatSPP. (FIG. 6).

Further to this, as shown in FIG. 7, Fluorometric assays were performed using cathepsins K, V, L and B (a-d, respectively) in the presence of predetermined concentrations of the rCatSPP. Fluorescence was monitored for 30 mins and the RFU plotted over time to generate fluorometric progress curves. The apparent first order, rate, constants produced by the inhibition of the cathepsins by the rCatSPP were subjected to non-linear regression analysis (inset) enabling the determination of inhibition constants (Ki) as 17.6 nM (±1.3), 4.8 nM (±0.6), and 0.62 nM (±0.14), respectively. All fluorometric assays were performed in replicates of three.

With establishment of anti-peptidolytic activity confirmed, the inventors then used the rCatSPP to demonstrate its ability to block the elastinolytic activity of CatS. The fluorogenic substrate Elastin-DQ was used to monitor the elastinolytic turnover of CatS in the presence of CatSPP (5.0-500 nM) over a 60 minute incubation and the inventors were able to demonstrate inhibition of this activity (FIG. 8).

The expression of CatS, L, K and V in four human malignant cell lines was evaluated by RT-PCR. Each of the cathepsins appears to be expressed in the four cell lines and amplification of Actin was used as an internal control (FIG. 9).

Based on the full peptidolytic inhibition profile calculated for the rCatSPP, and evidence that elastinolytic activity of at least CatS could be shown, the inventors proceeded to analyse the effectiveness of the peptide to block the activity of these proteases in invasive cancer models. For these experiments the inventors employed studies examining the invasion of tumour cells through matrigel coated modified Boyden chambers, (Flannery et al., 2003). These experiments were carried out on cell lines representative; of common types of cancer. Specifically these were PC3 (prostate cell line), HCT 116 (colorectal), U251MG (astrocytoma) MDA-MB-231 (breast) and MCF7 (breast), and results are shown in FIG. 10. FIG. 10 a illustrates the analysis of four human malignant cell lines by in vitro invasion assay; a-d: HCT116, U251 mg, MDA-MB-231 and PC3. Each cell line showed significant reduction in tumour cell invasion in the presence of the CatSPP. (*=p: ≦0.01, **=p: ≦0.001, ***= p: ≦0.0001). All variables were performed in triplicate with ten digital images captured and analysed for tumour cell invasion. The standard errors were plotted as ±error of the mean. Statistical significance was calculated using the students t-test. FIG. 10 b shows a histogram illustrating significant reduction (63%) in MCF7 tumour cell invasion in the presence of the CatSPP

An MTT assay was performed to assess the cytotoxic or proliferative effects of the rCatSPP protein. The MTT assay was performed using HCT116 colorectal carcinoma cells, incubated with 200 nM of the rCatSPP, control protein and vehicle-only control. The results (FIG. 11) illustrate that the recombinant protein has no significant effect on cell growth. All variables were repeated in quintuplet.

The inventors proceeded to investigate the effect of providing ah Fc portion on the cathepsin propeptide on the inhibition of L-type cathepsin protease in invasive cancer models.

A cathepsin S propeptide comprising a C-terminal Fc portion (CatSPP Fc) was cloned and expressed using the methods as described for CatSPP above. The cDNA sequence of the CatSPP was cloned into the pRSET A-Fc vector. A selection of 8 colonies from the positively transformed plate was subjected to colony PCR analysis using vector specific primers. All 8 colonies appear positive due to the amplification, of a band of approximately 1100 bp (FIG. 12)

The expression of the rCatSPP-Fc from the pRSET A vector was induced by the addition of IPTG. The results are shown In FIG. 13. Samples in lanes A, B, C and D contain uninduced and induced (B=0.2, C=0.5 and D=0.7 OD (A550 nm) respectively); Samples were resolved by SDS-PAGE and western blotting performed using an anti-polyhistidine tag antibody. The His-tagged protein species with a molecular weight of approximately 46 kDa was detected, equivalent to the predicted size of rCatSPP-Fc. The induction of expression in the culture with an OD of 0.2 appeared most optimal for protein production.

The rCatSPP-Fc was purified using MAC by virtue Of its N-terminal His-tag. The results are shown in FIG. 14 a) The purification profile shows two distinct peaks, a sharp peak at after approximately 200 mins and a second broader peak between 225 and 250 mins. b) The analysis of eluted fractions from the purification suggests that the first peak represents elution of non-specifically bound proteins from the column (fractions 1-5), whereas the broad secondary peak shows elution of a species of approximately 46 kDa, in agreement with the expected size of the rCatSPP-Fc (fractions 6-15). c) Analysis of purification fractions by western blotting using an anti-polyhistidine tag monoclonal antibody shows the presence of a his-tagged species of approximately 46 kDa as expected for the rCatSPP-Fc.

The inhibition of CatS peptidolytic activity using CatSPP Fc was measured. The rCatSPP-Fc was assessed by fluorometric assay using the fluorogenic substrate Z-VVR-AMC to determine if the species had the ability to retain its inhibition of CatS after the addition of the Fc-domain without any negative effects on the kinetics. The results are shown in FIG. 15: a) Progress curves demonstrate the imhibition of CatS activity in the presence of increasing concentrations of the rCatSPP-Fc (0 nM to 200 nM). a, inset) The Fc-control protein (200 nM) had no discernable effects on CatS activity, b) Rates were extrapolated from the progress curves and the kinetic of the inhibition were calculated as 8.9 nM (±2.5). Assays were repeated three times.

The stability of CatS PP versus CatS PP-Fc was assessed as follows. The rCatSPP and rCatSPP-Fc proteins were incubated with HGT116 colorectal carcinoma cells to assess the stability of the recombinant proteins by addition of the antibody IgG₂ Fc-domain. Samples of supernatant were assessed by western blotting (FIG. 16) to determine stability within the cell supernatant. The rCatSPP can only be detected at 0 hr whereas stability of the rCatSPP-Fc appears improved, due to its detection after 24 hrs; As controls, cell supernatants containing no added protein (−) were also assessed and membranes were stained with Ponceau Red to confirm equal loading of supernatants. Experiments were performed in triplicate.

The effect of the CatSPP Fc on cathepsin S in an in vitro invasion assay using prostate PC3 cells was then tested. The results are shown in FIG. 17, The histogram shows a quantitative summary of the PC3; invasion assay in the presence of CatSPP Fc (0-32 nM); Each assay was performed in triplicate and ten fields were counted in each assay.

The effect of the CatSPP Fc on cathepsin S in an in vitro invasion assay using other tumour cell lines was then tested (FIG. 18) The rCatSPP and rCatSPP-Fc proteins were applied to in vitro invasion assays using the HCT116 colorectal cell line. Assays were performed in the presence of increasing concentrations of the rCatSPP (0 nM to 250 nM) or rCatSPP-Fc (0 nM to 50 nM) and also appropriate control proteins at the maximal concentration. Standard deviations are plotted as error bars. Assays were repeated in triplicate with ten images captured from each. The standard deviation in mean tumour cell invasion is plotted as ±error bars.

Similar results were found in an invasion assay conducted using MDA-MB-231 cells. FIG. 19 illustrates relative EC50 values for rCatS PP and rCatS PP-Fc. The relative rate of MDA-MB-231 tumour cell invasion in the presence of varying concentrations of the rCatSPP or rCatSPP-Fc were subjected to non-linear regression analysis and sigmoidal dose-response curves constructed. The resultant EC50 values were found to be 78.0 nM and 8.3 nM for the (a) rCatSPP and (b) rCatSPP-Fc respectively.

As can be seen, CatSPP Fc acted as a potent inhibitor of the cathepsin S in the invasion assays, with the maximum inhibition being significantly greater and the inhibitory concentration being significantly less than that produced with CatSPP with no Fc portion. Although, it may have been expected that the stability of the CatSPP molecule would be enhanced to a small extent by the Fc portion, it is nevertheless very surprising that the inclusion of the Fc portion so significantly enhanced the inhibitory effect. Thus, the results demonstrate that the inclusion of an Fc portion with a cathepsin propeptide enhances the inhibition of the activity of cathepsin L-type protease in tumour invasion models.

Other studies have been performed using broad spectrum small molecule inhibitors of cathepsins in tumorigenesis models, demonstrating similar effects. Joyce and colleagues employed the use of JPM-OEt, an cell permeable analogue of the broad spectrum cysteine cathepsin inhibitor E64, in studies oh transgenic RIP-Tag2 mice (Joyce et al, 2004). These mice develop pancreatic islet tumours at 12-14 weeks due to the presence of the oncogenic SV40 T antigen. They demonstrated that the administration of this broad spectrum inhibitor to these mice could significantly inhibit multiple stages of tumour development, including the development of highly invasive carcinomas upon histological analysis of the animals. In an another investigation, Flannery and co-workers examined the use of 4-morpholineurea-Leu-homoPhe-vinylsulfone (LHVS), in blocking astrocytoma invasion. Using the same invasion model as the inventors have employed here, it was demonstrated that LHVS, which potently inhibits CatS and to a lesser extent Cat could block U251MG cells invading at up to 60% at a 50 nM concentration (Flannery et al, 2003). Collectively, these previous studies clearly demonstrate firstly the role that CatL-like proteases play in invasion processes in these cell lines, and secondly their potential as therapeutic targets for cancer therapeutics.

Despite considerable research efforts, the extent to the role of each of these proteases in tumorigenesis is yet to be fully appreciated. Clearly a substantial amount of evidence points towards their role in the breakdown of elastin, collagen and other components of the extracellular matrix, once they have been secreted by the tumorigenic cells. However, the role these enzymes play in the activation and control of each other and other, less closely related proteases, such as the metalloproteases is now emerging (Kobayashi et, al., 1993). Moreover, new evidence has come to highlight the role of CatS in the breakdown of matrix-derived anti-angiogenic factors, and production of pro-angiogenic factors during tumour progression (Wang et al, 2005). This demonstrates that these proteases could have more roles than simply the digestion of surrounding ECM to allow progression and migration of tumour.

### CONCLUSIONS

Here the inventors have described a novel expression and purification method for the production of cathepsin propeptides, for example CatSPP. The inventors have demonstrated that, by inhibition of cathepsin L-type proteases using cathespin propeptides, for example rCatSPP, tumorigenesis may be attenuated. Given the inhibition profiles that the inventors have seen in in vitro invasion assays using a range of different tumour cell lines, it is clear that the broad inhibition of the CatL-like proteases has clear therapeutic benefit to the clinical treatment of cancer. The ability to develop agents that can block the spread of tumours, particularly to secondary sites in the body would be attractive to the co-administration of cytotoxic agent regimes. The ability to rapidly produce the rCatSPP from bacterial cultures and apply it successfully in these tumour invasion models suggest that it could represent a novel approach to the design of therapeutic protease inhibitors.

All documents referred to in this specification are herein incorporated by reference. Various modifications and variations to the described embodiments of the inventions will be apparent to those skilled in the art without departing from the scope and Spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled iii the art are intended to be covered by the present invention.

### REFERENCES

Abrahamson et al, (2003) Biochem Soc Symp. 70, 179-99.
Adachi et al, 1998 September; 39(10):1789-96.
Bevec et al, J Exp Med. 1996 Apr. 1; 183(4):1331-8.
Broker et al, (2005). Cell death independent of caspases: a review. Clin Cancer Res. 11, 3155-62.
Bromme et al Biochemistry. 1999 Feb. 23; 38(8):2377-85.
Bromme D, Kaleta J. (2002) Curr Pharm Des 8:1639-58.
Bromme et al Biochem J. 1996 Apr. 1; 315 (Pt 1):85-9.
Bromme D, Okamoto K. Biol Chem Hoppe Seyler. 1995 June; 376(6):379-84.
Brubaker et al. 2003 February; 18(2):222-30.
Bryant P; Ploegh H. (2004). Curr Opin Immunol. 16, 96-102.
Buhling F, et al Adv Exp Med. Biol. 2000; 477:241-54.
Carmona et al (1996). Biochemistry. 35, 8149-57.
Chapman et al (1994 Am J Respir Crit Care Med. 150:S155-9.
Chapman H A et al (1997). Annu Rev Physiol, 59, 63-88.
Chauhan et al J Biol. Chem. 1993 Jan. 15; 268(2):1039-45.
de Duve C. (1983). Eur J Biochem 137:391-7.
Drake et al J Biol. Chem. 1996 May 24; 271(21): 12511-6.
Fehrenbacher N, Jäättelä M. (2005). Cancer Res 65, 2993-2995.
Flannery T, et al (2003). Am J Pathol. 163, 175-82.
Folkman J, Ingber D. (1992). Semin Cancer Biol. 3, 89-96.
Fox, T et al (1992) Biochemistry 31, 12571-12576.
Gal S, Gottesman M M. Biochem J. 1988 Jul. 1; 253(1):303-6.
Gelb et al Biochem Mol. Med. 1996 December; 59(2):200-6.
Gelb Science, 273, 1236-8.
Goulet et al Mol. Cell. 2004 Apr. 23; 14(2):207-19.
Guay J, et al (2000). Eur J. Biochem. 267:6311-8.
Hara K, et al FEBS Lett. 1988 Apr. 11; 231(1):229-31.
Honey K, Rudensky A Y. (2003). Nat Rev Immunol, 3, :472-82.
Hook V et al Biol. Chem. 385, 473-80.
Hummel K M, et al J Rheumatol. 1998 October; 25(10): 1887-94.
Inaoka T, et al Biochem Biophys Res Commun. 1995 Jan. 5; 206(1):89-96.
Itoh R, et al. DNA Res. 1999 Apr. 30; 6(2):137-40.
Joseph et al J Clin Invest. 1988 May; 81(5):1621-9.
Joyce J A, et al (2004). Cancer Cell. 5, 443-53.
Kafienah et al Biochem J. 1998 May 1; 331 (Pt 3):727-32.
Kargel H J et al Acta Biol Med. Ger. 1981, 40(9):1139-43.
Kirschke H, et al Biochem J. 1989 Dec. 1; 264(2):467-73.
Kobayashi et al (1993 Biochim Biophys Acta. 1178, 55-62
Koblinski J E et al (2000). Clin Chim Acta. 291, 113-35.
Lah T T, Kos J. (1998). Biol. Chem. 379:125-30.
Littlewood-Evans A J, et al Cancer Res. 1997 Dec. 1; 57(23):5386-90.
Mach et al (1994). J Biol. Chem. 269, 13036-40
Maubach C, et al (1997). Eur J. Biochem. 250, 745-50.
McGrath M E. (1999. Annu Rev Biophys Biomol Struct. 28, 181-204.
Morrison J F, Walsh C T. (1988). Adv Enzymol Relat Areas Mol. Biol. 61, 201-301.
Motyckova G and Fisher D E. (2002). Curr Mol. Med. 2002 August; 2(5):407-21.
Nakagawa T Y, et al. (1999); Immunity. 10, 207-17.
Nakagawa T, et al (1998 Science. 280:450-3.
Nishimura Y, et al Arch Biochem Biophys. 1988 Feb. 15; 261(1):64-71.
Ogrinc et al FEBS Lett. 1993 Dec. 28; 336(3):555-9.
Premzl A, et al. (2003) Exp Cell Res. 283, 206-14.
Rao J S. (2003), Nat Rev Cancer. 3, 489-501.
Rapa I, Volante M, Cappia S, Rosas R, Scagliotti G V and Papotti M. (2006). Cathepsin K is selectively expressed in the stroma of lung adenocarcinoma but not in bronchioloalveolar carcinoma. A useful marker of invasive growth.
Rawlings, et al (2004) Nucleic Acids Res. 32 Database issue, D160-D164
Reinheckel T, et al (2001). Biol Chem, 382, 735-41
Rescheleit D K, et al FEBS Lett. 1996 Oct. 7; 394(3):345-8.
Roth W, et al FASEB J. 2000 October; 14(13):2075-86.
Saftig P, et al Proc Natl Acad Sci USA. 1998 Nov. 10; 95(23): 13453-8.
Salminen A, Gottesman M M. Biochem J. 1990 Nov. 15; 272(1):39-44.
Saneshige S et al Biochem J. 1995 Aug. 1; 309 (Pt 3):721-4.
Santamaria I, et al Cancer Res. 1998 Apr. 15; 58(8):1624-30.
Seth P, et al Gene; 2003 Dec. 4; 321:83-91.
Shi G P, et al FEBS Lett. 1995 Jan. 3; 357(2):129-34.
Sinha D, et al (1994). Biotechniques. 17, 509-12, 514.
Sloane et al (1981) Science. 212, 1151-3.
Sloane et al (1994). J Cell Sci. 107, 373-84.
Tao K, et al (1994). Arch Biochem Biophys. 311, 19-27.
Tolosa E, et al J Clin Invest. 2003 August; 112(4):517-26.
Turk V et al (2004). Cancer Cell. 5, 409-10.
Wang et al (2005). J Biol. Chem. 2005 Dec. 19; [Epub ahead of print]
Wiederanders B. (2000). Adv Exp Med Biol 477:261-70.
Wiederanders B, Kirschke H. 1989 Aug. 1; 272(2):516-21,
Zheng T (2005). J. Immunol. 174:8106-15.

1. A method of inhibiting activity and/or overexpression of a cathepsin L-like protease in cells or tissue, said method comprising administration of a cathepsin propeptide or a nucleic acid encoding a cathepsin propeptide to said cells or tissue.

2. (canceled)

3. A method of treating a condition associated with aberrant activity and/or overexpression of a cathepsin L-like protease in a patient in need of treatment thereof, said method comprising administration of a cathepsin propeptide or a nucleic acid encoding a cathepsin propeptide.

4. The method according to claim 3 wherein the condition associated with aberrant activity and/or overexpression of a cathepsin L-like protease is a neoplastic disease, an inflammatory disorder, a neurodegenerative disorder, an autoimmune disorder, asthma, or atherosclerosis.

5. The method according to claim 1, wherein the cathepsin propeptide is a human cathepsin propeptide comprising amino acid sequence corresponding to amino acid residues 17 to 113 of the cathepsin S protease amino acid sequence shown in SEQ ID NO: 20. or the amino acid sequence corresponding to amino acid residues 13 to 109 of the amino acid sequence shown in SEQ ID NO: 13.

6. The method according to claim 1, wherein the cathepsin propeptide is a human cathepsin propeptide having the amino acid sequence shown in SEQ ID NO: 13, or the amino acid sequence shown in SEQ ID NO: 21.

7. The method according to claim 1, wherein the cathepsin propeptide comprises an antibody Fc portion.

8. The method according to claim 1, wherein the cathepsin L-like protease is cathepsin S.

9.-11. (canceled)

12. The method according to claim 3, wherein the cathepsin propeptide is a human cathepsin propeptide comprising amino acid sequence corresponding to amino acid residues 17 to 113 of the cathepsin S protease amino acid sequence shown in SEQ ID NO: 20, or the amino acid sequence corresponding to amino acid residues 13 to 109 of the amino acid sequence shown in SEQ ID NO: 13.

13. The method according to claim 3, wherein the cathepsin propeptide is a human cathepsin propeptide having the amino acid sequence shown in SEQ ID NO: 13, or the amino acid sequence shown in SEQ ID NO: 21.

14. The method according to claim 3, wherein the cathepsin propeptide comprises an antibody Fc portion.

15. The method according to claim 3, wherein the cathepsin L-like protease is cathepsin S.

16.-21. (canceled)

22. A pharmaceutical composition comprising a cathepsin propeptide or a nucleic acid encoding a cathepsin propeptide.

23. The pharmaceutical composition according to claim 22, wherein the cathepsin propeptide is a human cathepsin propeptide comprising amino acid sequence corresponding to amino acid residues 17 to 113 of the cathepsin S protease amino acid sequence shown in SEQ ID NO: 20, or the amino acid sequence corresponding to amino acid residues 13 to 109 of the amino acid sequence shown in SEQ ID NO: 13.

24. The pharmaceutical composition according to claim 22, wherein the cathepsin propeptide is a human cathepsin propeptide having the amino acid sequence shown in SEQ ID NO: 13, or the amino acid sequence shown in SEQ ID NO: 21.

25. The pharmaceutical composition according to claim 22, wherein the cathepsin propeptide comprises an antibody Fc portion.

26. The pharmaceutical composition according to claim 22, wherein the cathepsin L-like protease is cathepsin S.

27. A method for the recombinant production of cathepsin propeptides, said method comprising expressing a cathepsin propeptide with an N-terminal polyhistidine tag and purifying the expressed propeptide using metal ion affinity chromatography (IMAC).
